(19) United States
(12) Patent Application Publication
(10) Pub. No.: US 2022/0091128 A1
(43) Pub. Date: Mar. 24, 2022

(54) SYSTEM AND METHOD FOR LIGAND THERMAL ANALYSIS

(71) Applicant: Portland State University, Portland, OR (US)

(72) Inventors: Albert S. Benight, Milwaukie, OR (US); Matthew W. Eskew, Portland, OR (US)

(73) Assignee: Portland State University, Portland, OR (US)

(21) Appl. No.: 17/424,430

(22) PCT Filed: Jan. 21, 2020
(86) PCT No.: PCT/US2020/014364

§ 371 Date: Jul. 20, 2021

Related U.S. Application Data
(60) Provisional application No. 62/795,385, filed on Jan. 22, 2019.

(51) Int. Cl. G01N 33/68; G16H 50/20; G01N 25/48; G01N 33/545

(52) U.S. Cl. CPC G01N 33/6803; G01N 33/545; G01N 25/486; G16H 50/20

(57) ABSTRACT

Devices for ligand capture and methods of using the device are disclosed. The ligand may be captured from a sample, such as a plasma sample. Methods of identifying, quantifying, and/or characterizing captured ligands also are disclosed. Computer systems and methods for analyzing thermograms and determining the characteristics of ligands present in a sample are disclosed.

### CROSS-REFERENCE TO RELATED APPLICATION

This application claims the benefit of the earlier filing date of U.S. Provisional Application No. 62/795,385, filed Jan. 22, 2019, which is incorporated by reference in its entirety herein.

### FIELD

This disclosure concerns embodiments of a capture device for capturing ligands in a sample as well as methods of using the capture device. This disclosure also concerns embodiments of methods for identifying and/or quantifying ligands in samples. This disclosure also concerns embodiments of methods for identifying and/or characterizing new chemical entities in preclinical drug discovery.

### SUMMARY

A device for ligand capture includes (i) a body comprising a substrate material, wherein the body is an elongated body with a polygonal cross-section or wherein the body is an annular body; (ii) a poly(methyl methacrylate) (PMMA) coating on at least a portion of a surface of the body; and (iii) a plurality of retrieval moiety molecules covalently bound to the PMMA coating. In some embodiments, the substrate material comprises a ferromagnetic metal, a polymer, or glass.

In some embodiments, the body is an elongated body with a polygonal cross-section, an upper surface, a lower surface, and a plurality of side surfaces, and the PMMA coating is on at least one of the side surfaces. The polygonal cross-section may be cooperatively dimensioned to fit within a well of a 96-well plate or a neck of a vial or micro-centrifuge tube.

In some embodiments, the body is an annular body having an outwardly facing surface and an inwardly facing surface, and the PMMA coating is on at least a portion of the inwardly facing surface. The annular body may have an outer diameter less than an inner diameter of a well of a 96-well plate or less than an inner diameter of a neck of a vial or micro-centrifuge tube. In certain embodiments, the device further includes an upper annular portion having an outer diameter greater than the inner diameter of the well or neck.

A method of using the disclosed device includes combining, in a vessel, a capture moiety and a plasma sample comprising or suspected of comprising a ligand, the capture moiety comprising biotin covalently attached to a protein capable of binding to the ligand; incubating the plasma sample and capture moiety whereby the ligand, if present, binds to the capture moiety to form a conjugate comprising the capture moiety and the ligand; and removing the conjugate from the plasma sample with a device as disclosed herein. In some embodiments, the device comprises the capture moiety, and combining the capture moiety and the plasma sample comprises inserting the device into the plasma sample, whereby the conjugate forms. In some embodiments, the protein of the capture moiety is a plasma protein. The method also may include removing the ligand from the device, combining the ligand with a quantity of plasma or a solution comprising one or more proteins to provide an analysis sample, wherein the plasma or the solution comprising one or more proteins is devoid of the ligand, and obtaining a thermogram of the analysis sample by differential scanning calorimetry (DSC). In some embodiments, the method further includes inputting the thermogram into a computer system; comparing, using the computer system, the thermogram of the analysis sample to (i) a thermogram of a control sample comprising the plasma or the solution comprising one or more proteins, wherein the plasma or the solution is devoid of the ligand, (ii) a reference library of thermograms of samples comprising known ligands and plasma, samples comprising known ligands in solutions comprising one or more proteins, or both (i) and (ii) to provide a comparison; and determining, using the computer system and based at least in part on the comparison, whether the ligand is present in the analysis sample.

In some embodiments, the plasma sample is obtained from a subject, and the method further comprises diagnosing the subject with a disease or condition based at least in part on the identity, the quantity, or the identity and the quantity of the ligand in the plasma sample. In some embodiments, the plasma sample is obtained from a subject, the ligand is an exogenous therapeutic compound, and the method further includes determining a bioavailability of the exogenous therapeutic compound or a half-life of the exogenous therapeutic compound in the subject based on a quantity of the exogenous therapeutic compound in the plasma sample and an administered dosage of the exogenous therapeutic compound.

In some embodiments, a drug discovery or analysis method includes (a) combining a quantity of a drug candidate with a quantity of a solution comprising one or more plasma proteins to provide an analysis sample; (b) obtaining a thermogram of the analysis sample by differential scanning calorimetry; (c) inputting the analysis sample thermogram into a computer system; (d) comparing, using the computer system, the analysis sample thermogram to a thermogram of a control sample comprising the solution comprising one or more plasma proteins to provide a comparison, the control sample being devoid of the drug candidate; (e) determining, based at least in part on the comparison, whether the analysis sample thermogram exhibits a perturbation; and (f) if a perturbation is exhibited, (i) repeating steps (a)-(e) with one or more additional quantities of the drug candidate; and (ii) determining, based at least in part on the perturbation, a characteristic of an interaction of the drug candidate with the one or more plasma proteins, wherein the characteristic is a binding constant, reaction enthalpy, binding stoichiometry, binding free energy, binding entropy, or any combination thereof.

Further disclosed is a non-transitory computer-readable medium storing instructions which, when executed by one or more processors, cause the processors to perform a method comprising: receiving an input sample record comprising a thermogram of a corresponding plasma sample and an identification of a ligand present in the plasma sample; and determining, using a trained machine learning model, a quantity or concentration of the ligand in the plasma sample. The instructions may further, when executed by the processors, cause the processors to incrementally grow the trained machine learning model using the determined quantity or concentration and at least part of the input sample record.

In some embodiments, a method of determining an identity or quantity of a ligand present in an unknown sample includes establishing a feature vector specification derived from a thermogram specification, clinical history attribute specifications, and chemical and/or physical analysis output specifications; obtaining a plurality of labeled feature vectors, according to the feature vector specification, corresponding to respective samples; training a selected machine learning model with at least a portion of the plurality of labeled feature vectors; obtaining an unlabeled feature vector, according to a proper subset of the feature vector specification, corresponding to an unknown sample; and applying the trained machine learning model to the unlabeled feature vector to determine an identity or a quantity of a ligand present in the unknown sample. In certain embodiments, the method for includes, subsequent to the applying, determining that the trained machine learning model is inapplicable to a second sample; performing chemical and/or physical analysis on the second sample to obtain a second labeled feature vector, according to the feature vector specification, corresponding to the second sample; and incrementally growing the trained machine learning model using the second labeled feature vector.

The innovations can be implemented as part of one or more methods, as part of one or more computing systems adapted to perform an innovative method, or as part of computer-readable media storing computer-executable instructions that cause a computing system to perform the innovative method(s). The various innovations can be used in combination or separately.

The foregoing and other objects, features, and advantages of the invention will become more apparent from the following detailed description, which proceeds with reference to the accompanying figures.

### BRIEF DESCRIPTION OF THE DRAWINGS

FIGS. 1A and 1B are a front perspective view and a side view, respectively, of an exemplary capture device as disclosed herein.

FIG. 2 is a schematic diagram of another exemplary capture device as disclosed herein.

FIG. 3 is a perspective view of another exemplary capture device as disclosed herein.

FIG. 4 is a cross-sectional view of the capture device of FIG. 3.

FIG. 5 is a schematic cross-sectional view of the capture device of FIG. 3 in use.

FIG. 6 is a block diagram illustrating the use of a trained machine learning model to determine a label for a sample, and further illustrating incremental training of the machine learning model.

FIG. 7 is a block diagram illustrating training of a machine learning model.

FIG. 8 illustrates a generalized example of a suitable computing environment in which described embodiments, techniques, and technologies pertaining to a disclosed file index can be implemented.

FIGS. 9A and 9B are flowcharts illustrating two exemplary processes for capturing a ligand using a capture device as disclosed herein.

FIGS. 10A and 10B are thermograms showing the effects of 100 μM naproxen (NAP) and 100 μM bromocresol green (BCG) on plasma and HSA, respectively.

FIGS. 11A and 11B, respectively, show the effects of NAP and BCG interactions with human serum albumin (HSA) on thermodynamic parameters as a function of increasing ligand concentrations.

FIGS. 12A and 12B, respectively, show the effects of NAP and BCG interactions with HSA after capture and retrieval using various washes.

FIG. 13 is a flowchart of an exemplary general process for building a database.

FIG. 14 is a flowchart of an exemplary general process for drug development, therapeutic monitoring, and patient health status monitoring.

FIG. 15 is a flowchart of an exemplary machine learning model.

FIG. 16 is a flowchart of an exemplary process for developing a relational database.

FIG. 17 is a flowchart of an exemplary process for scoring clinical samples.

FIG. 18 is a flowchart illustrating exemplary drug development and clinical monitoring processes using a relational database.

FIGS. 19A-19E are thermograms showing the effects of 2 mg/mL NAP (19A), BCG (19B), DM1 (19C), tetracaine (Tet) (19D), and chloroquine (CQ) (19E) interactions with plasma.

FIGS. 20A and 20B show dose response curves for NAP, BCG, CQ, DM1, and Tet interactions with HSA. FIG. 20A shows Tm versus drug concentration, and FIG. 20B shows ΔG_{cal}(37° C.) versus drug concentration.

FIGS. 21A-21C are photographs showing gel electrophoresis of DNA capture. FIG. 21A shows gel results for an isolated 25-base single-strand DNA molecule; FIG. 21B shows gel results for an isolated 25 base pair cy5-labeled double-strand DNA molecule; FIG. 21C is a close-up of lanes 3-7 of FIG. 21B with contrast correction and enhancement to remove interference from DNA standard bands in lanes 1-2.

FIGS. 22A-22D are thermograms plotting baseline corrected μW versus temperature for thermograms of plasma alone (▪) and 25 base pair ssDNA alone (●) (22A); measured thermogram of plasma and ssDNA (▪) and thermogram calculated from the sum of the individual thermograms of plasma and ssDNA in FIG. 22A (●) (22B); thermograms of plasma alone (▪) and 25 base pair dsDNA alone (●) (22C); measured thermogram of plasma and dsDNA (▪) and thermogram calculated from the sum of the individual thermograms of plasma and dsDNA in FIG. 22C (●) (22D).

FIGS. 23A-23D are thermograms plotting baseline corrected μW versus temperature for thermograms of HSA_{B} alone (▪) and 25 base pair ssDNA alone (●) (23A); measured thermogram of HSA_{B} and ssDNA (▪) and thermogram calculated from the sum of the individual thermograms of HSA_{B} and ssDNA in FIG. 23A (●) (23B); thermograms of HSA_{B} alone (▪) and 25 base pair dsDNA alone (●) (23C); measured thermogram of HSA_{B} and dsDNA (▪) and thermogram calculated from the sum of the individual thermograms of HSA_{B} and dsDNA in FIG. 23C (●) (23D).

FIGS. 24A and 24B are graphs showing the effects of HSA biotinylation (HSA_{B}) on ligand binding. FIG. 24A shows standard HSA bound with NAP (▪), HSA_{B 1:1} with NAP (●), HSA_{B 1:5} with NAP (▴), and HSA_{B 1:10} with NAP (▾). FIG. 24B shows standard HSA bound with BCG (▪), HSA_{B 1:1} with BCG (●), HSA_{B 1:5} with BCG (▴), and HSA_{B 1:10} with BCG (▾).

FIGS. 25A and 25B are graphs showing the effects of pH on ligand binding to HSA. FIG. 25A shows standard HSA bound with NAP at pH 7.4 (▪), HSA with NAP at pH 8 (●), HSA with NAP at pH 6 (▴), and HSA with NAP in the presence of 50 μM BCG (▾). FIG. 25B shows standard HSA bound with BCG at pH 7.4 (▪), HSA with BCG at pH 8 (●), HSA with BCG at pH 6 (▴), and HSA with BCG in the presence of 50 μM NAP (▾).

FIGS. 26A and 26B are graphs showing two-ligand binding to HSA. FIG. 26A shows NAP binding in the presence of BCG: HSA+NAP (▪), HSA+25 μM BCG+NAP (●), HSA+50 μM BCG+NAP (▴), HSA+75 μM BCG+NAP (▾), and composite (additive) ΔG^{O}₃₇ values for NAP and BCG alone plus HSA (+). FIG. 26B shows BCG binding in the presence of varying amounts of NAP: HSA+BCG (▪), HSA+25 μM NAP+BCG (●), HSA+50 μM NAP+BCG (▴), HSA+75 μM NAP+BCG (▾), and composite (additive) G^{O}₃₇ values for NAP and BCG alone plus HSA (+).

FIG. 27 is a thermogram demonstrating a pH-dependent thermogram of HSA; (▪) HSA at pH 7.4; (●) HSA at pH 3; (Δ) HSA at pH 3 returned to ˜pH 7.

FIG. 28 is a plot showing a comparison of measured to literature binding constants for 19 drugs to HSA.

### DETAILED DESCRIPTION

Embodiments of a capture device for capturing ligands in a plasma sample, as well as embodiments of methods for using the capture device, are disclosed. This disclosure also concerns embodiments of methods for identifying and/or quantifying ligands in plasma samples. This disclosure also concerns embodiments of methods for identifying and/or characterizing new chemical entities in preclinical drug discovery.

### I. General Considerations, Definitions and Abbreviations

The following explanations of terms and abbreviations are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. Further, the term “coupled” encompasses mechanical, electrical, magnetic, optical, as well as other practical ways of coupling or linking items together, and does not exclude the presence of intermediate elements between the coupled items. The term “or” refers to a single element of stated alternative elements or a combination of two or more elements, unless the context clearly indicates otherwise. Furthermore, as used herein, the term “and/or” means any one item or combination of items in the phrase.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods as known to those of ordinary skill in the art. When directly and explicitly distinguishing embodiments from discussed prior art, the embodiment numbers are not approximates unless the word “about” is recited.

Although there are alternatives for various components, parameters, operating conditions, etc. set forth herein, that does not mean that those alternatives are necessarily equivalent and/or perform equally well. Nor does it mean that the alternatives are listed in a preferred order unless stated otherwise.

The systems, methods, and apparatus described herein should not be construed as being limiting in any way. Instead, this disclosure is directed toward all novel and non-obvious features and aspects of the various disclosed embodiments, alone and in various combinations and subcombinations with one another. The disclosed systems, methods, and apparatus are not limited to any specific aspect or feature or combinations thereof, nor do the disclosed things and methods require that any one or more specific advantages be present or problems be solved. Furthermore, any features or aspects of the disclosed embodiments can be used in various combinations and subcombinations with one another.

Although the operations of some of the disclosed methods are described in a particular, sequential order for convenient presentation, it should be understood that this manner of description encompasses rearrangement, unless a particular ordering is required by specific language set forth below. For example, operations described sequentially can in some cases be rearranged or performed concurrently. Moreover, for the sake of simplicity, the attached figures may not show the various ways in which the disclosed things and methods can be used in conjunction with other things and methods. Additionally, the description sometimes uses terms like “analyze,” “apply,” “build,” “determine” “display,” “estimate,” “generate,” “identify,” “instruct,” “obtain”, “produce,” “receive”, “train” to describe the disclosed computer-implemented methods. Such terms are high-level abstractions of the actual operations that are performed. The actual operations that correspond to these terms can vary depending on the particular implementation and can be readily discerned by one of ordinary skill in the art.

Theories of operation, scientific principles, or other theoretical descriptions presented herein in reference to the apparatus or methods of this disclosure have been provided for the purposes of better understanding and are not intended to be limiting in scope. The apparatus and methods in the appended claims are not limited to those apparatus and methods that function in the manner described by such theories of operation.

Definitions of common terms in chemistry may be found in Richard J. Lewis, Sr. (ed.), Hawley's Condensed Chemical Dictionary, published by John Wiley & Sons, Inc., 2016 (ISBN 978-1-118-13515-0). Definitions of common terms in molecular biology may be found in Benjamin Lewin, Genes VII, published by Oxford University Press, 2000 (ISBN 019879276X); Kendrew et al. (eds.), The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341); and other similar references.

In order to facilitate review of the various embodiments of the disclosure, the following explanations of specific terms are provided:

Conjugate: Two or more moieties directly or indirectly coupled together. For example, a first moiety may be covalently or noncovalently (e.g., electrostatically) coupled to a second moiety. Indirect attachment is possible, such as by using a “linker” (a molecule or group of atoms positioned between two moieties).

Differential scanning calorimetry (DSC): DSC measures the difference in the amount of heat required to raise the temperature of a sample and a reference as a function of temperature. During a phase transition, such as when a protein “melts” or unfolds, the amount of heat required changes, thereby providing a characteristic melting curve (ΔCₚ (kcal/mol·° C.) vs temperature (° C.)) of the protein as temperature is increased, where Cₚ is specific heat for a constant pressure process. The temperature at which the phase transition occurs varies from protein to protein. When a ligand binds to the protein, the melting temperature (mid-point of the melting curve) and/or maximum ΔCₚ may be affected.

Ferromagnetic: Susceptible to magnetization by exposure to an applied magnetic field, which may persist after removal of the applied field.

Ligand: A molecule that binds to a target molecule.

Moiety: A moiety is a fragment of a molecule, or a portion of a conjugate.

Perturb/perturbation: As used herein, the terms perturb, perturbed, and perturbation refer to differences (e.g., peak shifts, peak height variations) between a sample thermogram and a control thermogram.

Polymer: A molecule of repeating structural units (e.g., monomers) formed via a chemical reaction, i.e., polymerization.

Soluble: Capable of becoming molecularly or ionically dispersed in a solvent to form a homogeneous solution. U.S. Pharmacopeia definitions: very soluble: more than 1000 mg/ml, freely soluble: 100-1000 mg/ml, soluble: 30-100 mg/ml, sparingly soluble: 10-30 mg/ml, slightly soluble: 1-10 mg/ml, very slightly soluble: 0.1-1 mg/ml, practically insoluble or insoluble: <0.1 mg/ml.

Thermogram: As used herein, the term “thermogram” refers to a melting curve of a plasma sample or a solution comprising one or more plasma proteins, the thermogram produced by differential scanning calorimetry

### II. Capture Device and Method of Using

Embodiments of a device for ligand capture, such as plasma ligand capture, include a body comprising a substrate material, a poly(methyl methacrylate) (PMMA) coating on at least a portion of a surface of the body, and a retrieval moiety covalently bound to at least a portion of the PMMA coating. In some embodiments, the substrate material comprises a ferromagnetic metal (e.g., ferromagnetic steel), a polymer, or glass.

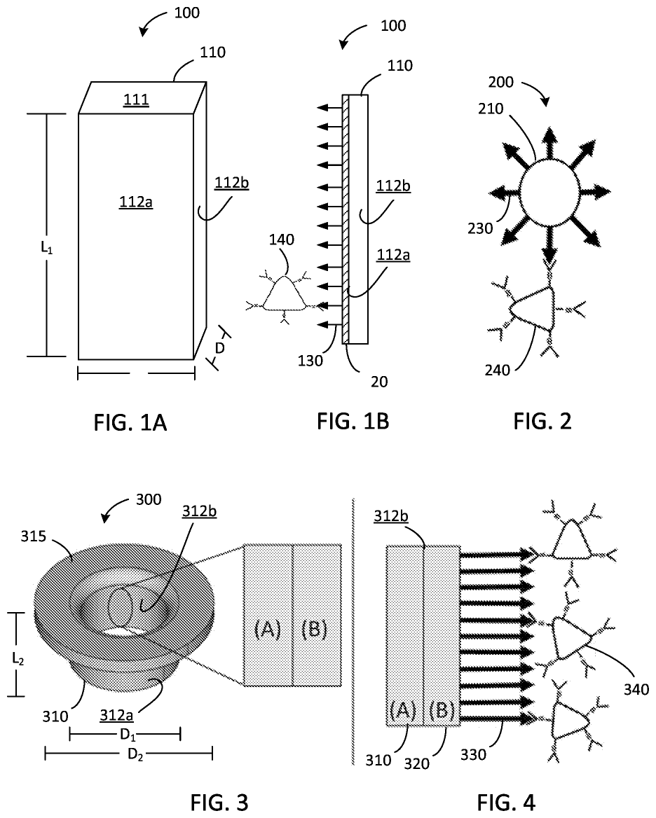

In some embodiments, as shown in FIG. 1A, a capture device 100 comprises an elongated body 110 having a length L₁ and a polygonal cross-section orthogonal to the length L. The body 100 has an upper surface 111, a lower surface (not visible in FIG. 1), and a plurality of side surfaces 112a, 112b, etc. Although the exemplary body 110 of FIG. 1A has a rectangular cross-section (see, e.g., upper surface 111), it is understood that the cross-section may be any polygon including three or more sides, e.g., a triangle, square, rectangle, parallelogram, trapezoid, pentagon, hexagon, octagon, or the like. Alternatively, the cross-section may be cylindrical or elliptical. As shown in FIG. 1B, the capture device 100 further includes a poly(methyl methacrylate) (PMMA) coating 120 on at least a portion of a surface (e.g., surface 112a) of the body 110, and a plurality of retrieval moiety molecules 130 bound to at least a portion of the PMMA coating 120. In some embodiments, the retrieval moiety comprises streptavidin molecules. The PMMA may be functionalized, e.g., by exposure to O₂ plasma, to create carboxylic groups to which streptavidin is subsequently attached. In any of the foregoing embodiments, a plurality of capture moieties 140 may be bound to at least some of the retrieval moieties 130. In certain embodiments, the capture moieties comprise a biotinylated protein, such as biotinylated human serum albumin (HSA).

In some embodiments, the capture device 100 has a polygonal cross-section cooperatively dimensioned to fit within a well of a 96-well plate or within a neck of a vial or a micro-centrifuge tube. A standard 96-well plate has wells with an inner diameter of 6.4 mm Thus, in certain embodiments, the device 100 has a polygonal cross-section that has a largest dimension of less than 6.4 mm With reference to FIG. 1A, in one embodiment, the device 100 has a rectangular cross-section having a width W and a depth D. In some examples, the width is within a range of 4-5 mm, the depth is within a range of 0.4-1 mm, and the length is within a range of 20-50 mm. The device 100 may be stamped from metal, or it may be manufactured using a polymer or glass material. In some embodiments, the entire body 100 is made of PMMA. In such embodiments, an additional PMMA coating is unnecessary.

In another embodiment (not shown), the capture device is a cap, such as a cap for a vial. The cap is constructed of metal and its interior surface is coated with streptavidin. In some examples, the metal is coated with PMMA, which is functionalized to create carboxylic groups to which the streptavidin molecules are attached. A plurality of capture moieties, such as biotinylated HSA, may be bound to the streptavidin molecules.

In some embodiments, as shown in FIG. 2, a capture device 200 comprises a bead 210. The bead may be constructed of a ferromagnetic material, e.g., ferromagnetic steel. In some embodiments, construction with a ferromagnetic metal facilitates movement and handling of the capture device 200 using a magnetic device. The capture device 200 further comprises a plurality of retrieval moiety molecules 130 bound to a surface of the bead 210. In certain embodiments, the bead 210 further comprises a PMMA coating (not shown) and the retrieval moieties are bound to the PMMA coating. In any of the foregoing embodiments, a plurality of capture moieties 240 may be bound to at least some of the retrieval moieties 230. In certain embodiments, the capture moieties comprise a biotinylated protein, such as biotinylated HSA.

In some embodiments, as shown in FIG. 3, a capture device 300 has an annular body 310. The annular body has an outwardly facing surface 312a, an inwardly facing surface 312b, an outer diameter D₁ and a length L₂. As shown in the cross-sectional view of FIG. 4, a PMMA coating 320 is disposed on at least a portion of the inwardly facing surface 311b. A plurality of retrieval moiety molecules 330, e.g., streptavidin molecules, is covalently bound to at least a portion of the PMMA coating 320. In certain embodiments, the device 300 further comprises an upper annular portion 315, the upper annular portion 315 having an outer diameter D₂ greater than the outer diameter D₁. In any of the foregoing embodiments, a capture moiety 340, e.g., a biotinylated protein, may be bound to the retrieval moiety molecules 330. The capture moiety and retrieval moiety may be collectively referred to as a capture reagent.

In any of the above embodiments, the outer diameter D₁ of the annular body 310 may be less than an inner diameter of a well of a 96-well plate or less than an inner diameter of a neck of a vial or micro-centrifuge tube. In any of the above embodiments, the outer diameter D₂ of the upper annular portion 315 may be greater than an inner diameter of a well of a 96-well plate or less than an inner diameter of a neck of a vial or micro-centrifuge tube. In some examples, the outer diameter D₁ is less than 6.4 mm, such as 3-5 mm, and the outer diameter D₂ may be 3-5 mm greater than the outer diameter D₁. In one example, the outer diameter D₁ is 4.9 mm, and the outer diameter D₂ is 7.9 mm. In any of the above embodiments, the capture device 300 may have a length L₂ within a range of from 2-40 mm, such as from 2.5-30 mm. When the capture device 300 will be used with a 96-well plate, the length L₂ may be 2-3 mm, such as 2.5 mm. When the capture device 200 will be used with a 2-mL vial, for instance, the length L₂ may be 5-30 mm, such as, 10-30 mm, 20-30 mm, 25-30 mm, or 28-29 mm. In any of the above embodiments, the capture device 300 may be constructed of a ferromagnetic metal, e.g., ferromagnetic steel. In some embodiments, at least the upper annular portion 315 is constructed of a ferromagnetic metal. In some embodiments, construction with a ferromagnetic metal facilitates movement and handling of the capture device 300 using a magnetic device, such as an automated sample handler.

### III. Methods of Identifying and/or Quantifying Ligands in Plasma

Human plasma is a complex fluid comprised of a variety of molecular cellular components constantly perfusing tissues throughout the entire body. Included in this process is distribution of exogenous therapeutic compounds and endogenous circulating components released in the interstitial fluid. Endogenous compounds might include metabolic and cellular degradation products that can be associated with health status. For example, in cancer, tumors constantly shed cell remnants releasing disease-specific proteins and protein fragments into plasma. In addition to being a transport medium for exogenous compounds, plasma contains an enormous repository of endogenous cellular components that can be directly reflective of collective physiological status and indicative of normal health.

The process of pre-clinical drug development efforts requires that new chemical entities (NCE) identified as potential drug candidates be both potent and bioavailable. To be potent an NCE must have specific and sufficient binding strength to its desired target. Bioavailability of the NCE requires the compound be properly absorbed, distributed, metabolized, excreted and not toxic or that it must possess favorable ADME/Tox characteristics. It is advantageous to gain a firm understanding of how the NCE interacts with the plasma proteome, including its interactions with human serum albumin (HSA). HSA is the primary component of plasma by mass and comprises 60% of the total protein in plasma. Central to its function, HSA binds a variety of ligands (peptides, proteins, lipids, etc.) and serves to circulate entities that bind it in the blood to various locations, organelles and organs throughout the body. Associated with these functions there are a multiplicity of reactive sites on HSA with a known binding affinity for a large variety of different ligands.

Embodiments of a method for identifying and/or quantifying ligands in plasma may be investigative and/or diagnostic in nature. The ligand may be any exogenous or endogenous molecule capable of binding to a protein, such as a plasma protein. In some embodiments, the ligand is a drug molecule.

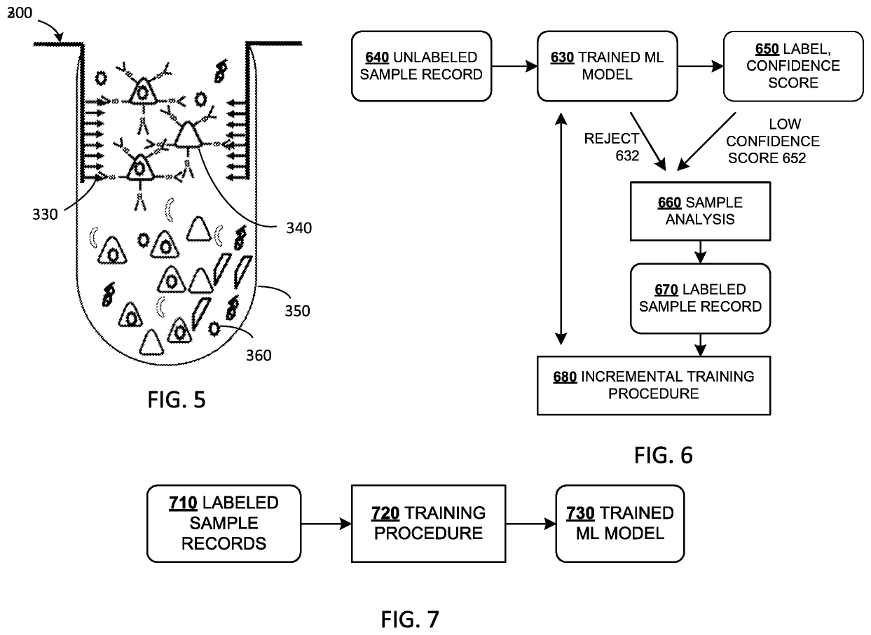

In some embodiments, (e.g., as shown in FIG. 5) the method includes combining, in a vessel 350, a capture agent or capture moiety 340 and a plasma sample comprising or suspected of comprising a ligand 360, the capture moiety 340 comprising biotin covalently attached to a protein capable of binding to the ligand 360; incubating the plasma sample and capture moiety 340 whereby the ligand 360, if present, binds to the capture moiety to form a conjugate; inserting a capture device, such as capture device 300 as disclosed herein, into the vessel, whereby the conjugate binds to the retrieval moiety 330 of the device 300; and removing the device 300 with the bound conjugate from the plasma sample. In certain embodiments, the capture device 300 comprises the capture moiety 340 and the retrieval moiety 330, and combining the capture moiety and plasma sample comprises inserting the capture device 300 into the vessel 350, whereby the ligand 360, if present, binds to the capture moiety 340 of the capture device to form a conjugate bound to the retrieval moiety 330.

The protein of the capture moiety may be any protein target. In some embodiments, the protein of the capture moiety is a plasma protein. In certain embodiments, the plasma protein is human serum albumin (HSA), IgG, fibrinogen, transferrin, haptoglobin, α-1-acid glycoprotein (α-AGP), complement C, or a combination thereof. In certain embodiments, the plasma protein is HSA. Advantageously, the capture moiety is selected based on its ability to bind to one or more ligands present, or suspected of being present, in the plasma.

The method further includes removing the bound ligand from the device. Various washing protocols may be followed to remove the bound ligand from the device. The removed ligand is combined with a quantity of plasma or a solution comprising one or more proteins (e.g., one or more plasma proteins) to provide an analysis sample. Advantageously, the plasma or the solution is devoid of the ligand. A thermogram of the analysis sample is obtained by differential scanning calorimetry (DSC).

DSC is useful for thermodynamic studies of protein denaturation. In DSC, excess heat capacity of temperature-induced unfolding of a protein sample is directly measured. Plasma thermograms measured by DSC are sensitive to mass, abundance, and effects of ligand (exogenous and endogenous) binding. In essence, plasma thermograms provide a system-wide snapshot of the status of the plasma proteome (and ligands therein) in terms of thermodynamic stability of the major plasma proteins and circulating ligands that bind them. As the most abundant plasma protein (60%) the fractional contribution of HSA comprises a significant portion of the overall signal making up the plasma thermogram. In some embodiments, a sample (e.g., HSA, HSA+ligand(s), or plasma in buffer) and a reference solution (the same buffer alone) are heated in tandem at the same rate while sample and reference temperatures are continually measured and compared. When molecular transitions occur in the sample, that are either exothermic or endothermic, the temperature of the sample is greater or less than that of the reference solution. At each temperature, the adjustment of power necessary to bring the sample temperature back to the reference temperature is directly proportional to the heat capacity, or change in specific heat, of the sample. The change in specific heat at constant pressure, ΔCₚ, is plotted as a function of temperature to produce a DSC melting curve or thermogram. Thermograms provide an evaluation of key thermodynamic parameters, i.e., enthalpy (ΔH_{cal}) and entropy (ΔS_{cal}) from which the free energy at 37° C., ΔG_{cal}(37° C.) of the melting process can be quantitatively evaluated. Characteristic features of the thermogram include: (1) temperature at the maximum peak height, Tm; (2) calorimetric enthalpy (ΔH_{cal}) evaluated form the integrated area under the DSC melting curve; and (3) the calorimetric entropy (ΔS_{cal}), which is closely related to the ratio (˜ΔH_{cal}/Tm). Relative values of thermodynamic values provide information on physical structural stability, chemical features, and ligand binding effects on the protein(s).

Thermograms measured by DSC are sensitive to interactions of ligands with plasma proteins, such as human serum albumin (HSA) and other less abundant plasma proteins. Observed perturbations of thermograms are highly sensitive to binding interactions, as well as structural modifications and/or isomerization. Generally, when a ligand recognizes and binds to a native protein, depending on the nature of the binding (electrostatic, polar, hydrophobic, etc.) it can either stabilize or destabilize that protein with respect to thermal and/or chemical denaturation. Consequently, relative to the protein in the absence of ligand, the melting temperature or denaturant concentration required to unfold the protein is either increased or decreased. Analogously, if a ligand were to selectively recognize some feature of a denatured protein, the melting temperature also could be altered. Temperature shifts on thermograms can be dramatic (easily tens of degrees) when a ligand binds to a protein, depending on the binding type and strength. Such effects produce characteristic patterns on thermograms that differ from an average or “normal” thermogram. In some embodiments, interactions with HSA are of particular interest. HSA can bind extraordinary levels of ligands, in some cases increasing the ligand's solubility in plasma by several fold. HSA is not only involved in transport of therapeutics, but can also significantly affect pharmacokinetics of administered therapeutics. Individual ligands, such as drugs, provide unique thermogram shifts, or signatures, that may be used to identify the presence or absence of a particular ligand in a sample. In some embodiments, the thermogram shift also provides quantitative data as the magnitude of the thermogram shift is related to the concentration of the ligand.

In any of the above embodiments, the thermogram may be inputted into a computer system (e.g., into a database of a computer system), and compared, using the computer system, to (i) a thermogram of a control sample comprising plasma or the solution comprising one or more proteins, wherein the plasma or the solution is devoid of the ligand, (ii) a reference library of thermograms of samples comprising known ligands and plasma, samples comprising known ligands in solutions comprising one or more proteins, or both (i) and (ii) to provide a comparison. The method further includes determining, using the computer system and based at least in part on the comparison, whether the ligand is present in the analysis sample. Presence of the ligand may be indicated by perturbations (e.g., shifts in position and/or magnitude of thermogram peaks) in the analysis sample thermogram relative to the control sample thermogram and/or by matching features (e.g., peak positions and/or peak magnitudes) of the analysis sample thermogram to reference thermograms of samples comprising known ligands. If the ligand is present, the method may further include determining, using the computer system and based at least in part on the comparison, an identity, a quantity, or an identity and a quantity of the ligand in the analysis sample. In some embodiments, the identity is determined by a peak position on the thermogram and/or a quantity is determined by a peak magnitude on the thermogram. In any of the above embodiments, a portion of the conjugate removed from the device may be analyzed further by chromatography, spectroscopy, gel electrophoresis, or a combination thereof to determine one or more properties (e.g., molecular weight, charge state, etc.) of the ligand.

The ligand may be an exogenous or endogenous ligand. Exogenous ligands may include, but are not limited to, small molecule therapeutic agents, such as drugs. For instance, a known quantity of a drug is administered to a subject. After a selected period of time, a plasma sample is obtained from the subject. The ligand (drug) is captured from the plasma sample as disclosed herein, and a quantity of the ligand in the plasma sample is determined. Such analysis may be used to determine bioavailability and/or circulating half-life of the drug.

Endogenous ligands include, but are not limited to, endogenous proteins, peptides, nucleotides, metabolites, fatty acids, phospholipids, steroids, disease-specific biomarkers, and the like. In some embodiments, the ligand is a biomarker associated with a disease state or medical condition. A plasma sample is obtained from a subject. The endogenous ligand is captured from the plasma sample as disclosed herein, and an identity and/or quantity of the endogenous ligand in the plasma sample is determined. The subject may be diagnosed with a disease or condition based at least in part on the identity and/or quantity of the endogenous ligand in the plasma sample.

In some embodiments, the method is a diagnostic or investigative method, wherein the method includes obtaining a plasma sample of a subject, the plasma sample comprising or suspected of comprising a ligand; obtaining a thermogram of the plasma sample by differential scanning calorimetry; comparing, using a computer system, the thermogram of the plasma sample to (i) a thermogram of a control sample comprising plasma or a solution comprising one or more plasma proteins, the control sample being devoid of ligands, (ii) a reference library of thermograms of samples comprising known ligands in plasma or the solution comprising one or more plasma proteins, or both (i) and (ii) to provide a comparison; and determining, using the computer system and based at least in part on the comparison, whether the ligand is present in the analysis sample. Presence of the ligand may be indicated by perturbations (e.g., shifts in position and/or magnitude of thermogram peaks) in the analysis sample thermogram relative to the control sample thermogram and/or by matching features (e.g., peak positions and/or peak magnitudes) of the analysis sample thermogram to reference thermograms of samples comprising known ligands. Perturbations in the thermogram may be indicative of infection, inflammation, malnutrition, autoimmune disease, and/or other diseases or conditions in the subject. If the ligand is present, the method may further include determining, using the computer system and based at least in part on the comparison, an identity, a quantity, or an identity and a quantity of the ligand in the plasma sample. In some embodiments, the identity is determined by a peak position on the thermogram and/or a quantity is determined by a peak magnitude on the thermogram. In certain embodiments, the ligand is a biomarker associated with a particular disease state or medication condition. In one embodiment, the method further comprises diagnosing the subject with a disease or condition based at least in part on the identity, the quantity, or the identity and the quantity of the ligand in the plasma sample. In an independent embodiment, the ligand is an exogenous therapeutic compound, and the method further comprises determining a bioavailability of the exogenous therapeutic compound or a half-life of the exogenous therapeutic compound in the subject based at least in part on a quantity of the exogenous therapeutic compound in the plasma sample and an administered dosage of the exogenous therapeutic compound.

In some embodiments, the method is an investigative method for preclinical drug discovery. A new chemical entity (NCE), or drug candidate, is combined with a quantity of plasma or a solution comprising one or more proteins to provide an analysis sample. A thermogram of the analysis sample is obtained by differential scanning calorimetry and inputted in to a database within a computer system. Using the computer system, the analysis sample thermogram is compared to a control sample comprising plasma or the solution comprising one or more proteins to provide a comparison, the control sample being devoid of the drug candidate. Based at least in part on the comparison, a determination is made regarding whether the analysis sample exhibits a perturbation. A perturbation indicates an interaction between the drug candidate and a protein in the plasma or the solution comprising one or more proteins. If a perturbation is present, the method may further include investigating the interactions between the drug candidate and particular proteins. The drug candidate is combined with a solution comprising one or more individual plasma proteins to provide a subsequent analysis sample. A thermogram of the subsequent analysis sample is obtained and inputted into the computer system. Using the computer system, the subsequent analysis sample thermogram is compared to a thermogram of a control sample comprising the solution comprising one or more plasma proteins to determine whether the subsequent analysis sample exhibits a perturbation. Advantageously, embodiments of the disclosed method include simple sample preparation and experimental execution, small sample volume (e.g., 500 μL), no required prior knowledge of binding parameters, and/or a short processing time (less than 90 minutes). Additionally, the method is fully amenable to automated, high-throughput and parallel screening applications.

In some embodiments, the data for each NCE is compared with those within individual classes of compounds already present in the database. From this comparison, specific binding characteristics of the NCE may be determined.

In any of the foregoing embodiments, the NCE may have poor water solubility. For example, the NCE may be slightly soluble, very slightly soluble, or insoluble in water. Conventionally, poorly soluble compounds are prepared in organic solvent, which is then serially diluted to a desired working concentration. However, an appropriate stock solution for further dilution generally requires a concentration at least 3 orders of magnitude higher than the presumed binding constant of the drug, which may be impractical. Moreover, the presence of residual organic solvent in the diluted solutions, even in minuscule amounts, can have significant effects on protein structure and subsequent ligand binding, thus confounding the screening results. Some poorly water-soluble compounds, however, are more soluble in the presence of an aqueous solution comprising a plasma protein than in water or aqueous buffer alone. In some embodiments, a stock solution of a poorly water soluble compound, such as an NCE, is prepared in a suitable organic solvent. An aliquot of the stock solution including a desired amount of the compound is evaporated under vacuum to provide the solid compound. A solution of one or more plasma proteins in aqueous buffer is added to the solid compound to provide an aqueous solution of the compound and the one or more plasma proteins that is suitable for thermogram analysis. In some embodiments, the one or more plasma proteins comprises HSA. In certain embodiments, the HSA-buffer solution has a concentration of 25-30 μM HSA.

In any of the foregoing embodiments, thermogram analysis in combination with the capture strategy may be a direct, fast, and simple means to provide a link between causative agents circulating in blood that bind to plasma proteins, and specific perturbations of plasma thermograms. Using the capture strategy, likely candidates can be isolated from plasma and their effects on a plasma or HSA thermogram independently assessed. By identifying circulating ligands in plasma, the capture approach provides a novel means to begin to unravel features of the molecular mechanism(s) underlying observed specific DSC plasma thermogram patterns, and their association with human disease and/or administered drugs.

Additionally, in some embodiments, the capture strategy may be an invaluable biomarker discovery and proteomics analysis screening tool. Embodiments of the disclosed capture strategy afford the ability to isolate retrieved material from plasma samples in sufficient quantities for extensive follow-on analysis including DSC. For example, effects of the retrieved, isolated material on an HSA thermogram directly demonstrate contributions of HSA/ligand interactions on an observed perturbed plasma thermogram. The degree to which the HSA thermogram is affected would define extent of the plasma thermogram perturbation that could be attributed to binding of HSA. Using this strategy would enable classification of important ligands based on their associated perturbations of the plasma thermogram. With sufficient amounts of data, the process provides relevant characterizations of captured ligands and predictions of their type and character based on their effects on plasma thermograms.

### IV. Machine Learning (ML) and Database

The disclosed technologies can be used to detect identities and/or quantities of ligands in a plasma sample directly from a plasma sample and, optionally, known sample characteristics, through the use of machine learning. Identification of a ligand can be treated as a classification problem. Determining ligand quantities (or equivalently, concentrations) can be treated as a regression problem. Determining both ligand identities and quantities can be treated as a regression problem, or as a combination of classification and regression. The identity or quantity or other parameter determined by a machine learning procedure is dubbed a “label”.

Generally, training data can be used to build a trained machine learning (ML) model for either classification or regression. Training data can be provided as a corpus of labeled sample records for training the ML model prior to deployment, or as individual labeled sample records subsequent to deployment in a learn-as-you-go approach, or as a combination.

A sample record is a record of multiple data fields pertaining to a sample, and can include one or more of: a thermogram of the sample, sample clinical history (e.g. patient or specimen characteristics, links to prior samples from the same patient or specimen, or any known treatments undergone by the patient, specimen, or sample). A sample record can additionally include any of a variety of chemical or physical analysis results for the sample, for example quantification or identification of analytes present, or data describing interactions between the analytes and plasma proteins, including thermodynamic interactions. A labeled sample record is a sample record for which the thermogram, ligand identities and quantities, and thermodynamic interaction between ligand and plasma protein are all known. An unlabeled sample record is a sample record for which at least one of these items is not known a priori, and is sought to be determined through application of the trained ML model.

By way of illustration, in a ligand identification application, an unlabeled sample record can include the thermogram and no direct knowledge of ligand identities, quantities, or thermodynamic interactions, and the trained ML model can be used to identify one or more ligands present in the sample. Alternatively, such an application can determine that all of a set of ligands, if present in the sample, are below respective threshold amounts; that is, a null result.

In an illustrative ligand quantification application as shown in FIG. 6, an unlabeled sample record 640 can include the sample thermogram and a priori knowledge of one or more ligands present in the sample, and a trained ML model 630 can be used to determine quantities of the known ligands (e.g. label 650). In a further application, a trained ML model can be used to determine both identities and quantities of one or more ligands.

For classification, a variety of ML approaches can be used, including, without limitation: linear or quadratic classifiers, support vector machines, kernel estimators (such as k Nearest Neighbors), decision trees, random forests, shallow or deep neural networks, of learning vector quantization. For regression, a variety of ML approaches can be used, including, without limitation: linear or multivariate regression, support vector machines, decision trees, random forests, shallow or deep neural networks, or Lasso regression. Principal components analysis (PCA), independent component analysis (ICA), or multiple discriminant analysis (MDA) can also be employed, particularly in applications where a sample can include multiple ligands.

In alternative embodiments, unsupervised learning can be employed, for example to associate samples of a population with clusters.

For training, as illustrated in FIG. 7, a labeled sample record 710 is mapped to a feature vector, each element of which can be, e.g., a binary, categorical, or continuous variable. In some examples, the sample record can itself be the feature vector. A thermogram can be characterized by a feature sub-vector of ordinate values (e.g. differential specific heat, ΔCp, or a similar thermodynamic variable) for respective abscissa values (e.g. temperature), or features derived from the thermogram (e.g. peak position, peak amplitude, peak width, peak asymmetry, maximum slope, tail area, a moment, kurtosis, peak separation, percentiles, or similar features derived from a derivative or integral of the thermogram). The feature vector can include features derived from the sample clinical history, or from chemical or physical analysis of the sample. An ML model can be selected and configured according to the structure of the feature vector. The available labeled sample records can be split into training and test datasets. The ML model can be trained using the training dataset and a training procedure 720 for the selected ML model, to obtain a trained model 730. Evaluation of the trained ML model can be performed using the test dataset. In some embodiments, hyper-parameters can be used and adjusted to improve the performance of the training procedure, as reflected in the performance of the trained ML model on the test dataset.

As shown in FIG. 6, the trained ML model 630 can be deployed and applied to unlabeled sample records 640, to determine identities and/or quantities of ligands in a plasma sample (e.g. label 650). That is, the trained ML model can take an unlabeled sample record as an input and provide one or more labels (together with at least a sample identifier) as an output. Alternatively, the trained ML model can take an unlabeled sample record as an input and provide a corresponding labeled sample record as an output. The ML model can also provide a confidence score 650 associated with its results for the sample.

In some embodiments, training can be continued after deployment of the ML model using an incremental learning approach. Incremental learning is well suited to ML models based on neural networks or decision trees, but can also be applied with other types of ML models. An unlabeled sample record can be provided to a trained or partially trained ML model. If the ML model is unable to make a determination from the sample record (reject 632), or if the ML model provides a determination with a confidence score below a threshold (652), then the sample can be sent for offline physical/chemical analysis 660 to generate a corresponding labeled sample record 670 for the same sample, which can then be applied in an incremental learning procedure 680 to update or grow the trained ML model 630.

In some examples, unlabeled sample records which do result in a satisfactory decision from the trained ML model can also be used to incrementally reinforce the ML model, for example if the confidence score is above a threshold.

The ML model can be coupled to one or more databases, including a relational database. In some examples, labeled sample records, or their associated feature vectors can be maintained as a database. In further examples, operations of the ML model on incoming unlabeled or labeled sample records, including label determinations or confidence scores, can be logged to a database. In additional examples, coefficients or parameters of the trained ML model (such as neural network coefficients) can be maintained in a database.

In some embodiments, the database stores data collected on a plurality of examined ligands. The database provides a foundational basis for comparative analysis of unknown compounds and new compounds. As analytical results for new ligands are obtained, they are added to the database. When sufficiently populated, the database can be used for virtual screening and enable grouping and comparisons of compounds according to their thermodynamic characteristics and properties. Embodiments of the database are dynamic, relational, and predictive.

In any of the foregoing embodiments, each sample stored in the database has a multifactorial vector associated with it, the vector comprising specific individual information and results for the sample (all collected data and metadata). Thermodynamic and binding parameters determined from measurements allow distillation of data in the form of standard drug interaction parameters including, but not limited to, binding stoichiometry, n, binding constant, K_{B}, saturation point, number of implied binding sites on the protein, free energy of binding, and combinations thereof. Within the database, these results are paired with metadata for each sample. In some embodiments, a drug's class (chloroquine, sulfa drug, etc.), known characteristics (e.g., molecular weight), and significant structural features, if available, are also associated with the drug in the database.

The disclosed technology can be provided as a service to a customer, wherein the customer provides either an unknown sample or a thermogram thereof, together with associated clinical history data and/or other sample data, and receives in return identification and/or quantities of one or more ligands present in the unknown sample. The disclosed technology can also be provided as software, in the form of non-transitory computer-readable media, wherein a single party (or related parties) provides the samples and thermograms, operates the trained machine learning model to determine quantities or identities of ligands in samples, and uses the results to support an application such as drug discovery or pre-clinical trials. The disclosed technology can also be provided as a system comprising a combination of computing hardware and software.

### V. Example Computing Environment

FIG. Error! Reference source not found. illustrates a generalized example of a suitable computing environment Error! Reference source not found.00 in which described examples, techniques, and technologies, including for determining identities or quantities of ligands in a sample, can be implemented. For example, the computing environment Error! Reference source not found.00 can implement all of the computer-implemented functions described herein. Particularly, the computing environment can implement training of a machine learning model or deployment of the trained machine learning model.

The computing environment Error! Reference source not found.00 is not intended to suggest any limitation as to scope of use or functionality of the technology, as the technology can be implemented in diverse general-purpose or special-purpose computing environments. For example, the disclosed technology can be implemented with other computer system configurations, including hand held devices, multiprocessor systems, microprocessor-based or programmable consumer electronics, network PCs, minicomputers, mainframe computers, and the like. The disclosed technology can also be practiced in distributed computing environments where tasks can be performed by remote processing devices that can be linked through a communications network. In a distributed computing environment, program modules can be located in both local and remote memory storage devices.

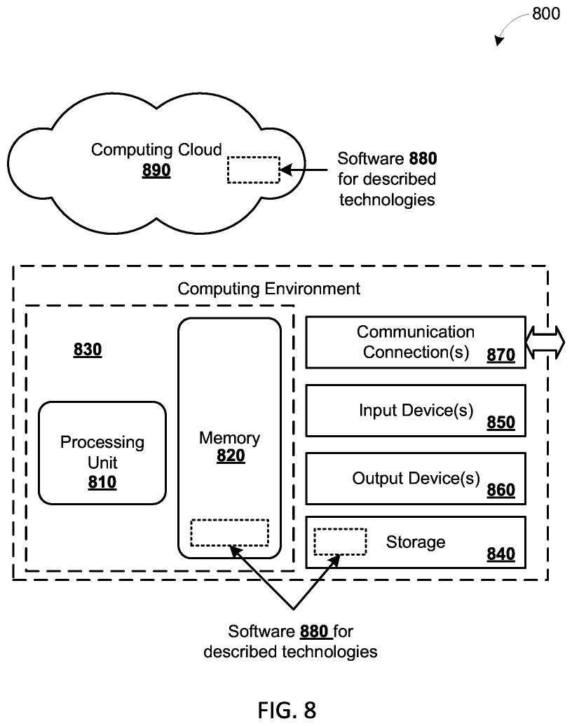

With reference to FIG. 8Error! Reference source not found., the computing environment 800 includes at least one central processing unit 810 and memory 820. In FIG. 8, this most basic configuration 830 is included within a dashed line. The central processing unit 810 executes computer-executable instructions and can be a real or a virtual processor. In a multi-processing system, multiple processing units execute computer-executable instructions to increase processing power and, as such, multiple processors can be running simultaneously. The memory 820 can be volatile memory (e.g., registers, cache, RAM), non-volatile memory (e.g., ROM, EEPROM, flash memory, etc.), or some combination of the two. The memory 820 stores software 880, images, and video that can, for example, implement the technologies described herein. A computing environment can have additional features. For example, the computing environment 800 includes storage 840, one or more input devices 850, one or more output devices 860, and one or more communication connections 870. An interconnection mechanism (not shown) such as a bus, a controller, or a network, interconnects the components of the computing environment 800. Typically, operating system software (not shown) provides an operating environment for other software executing in the computing environment 800, and coordinates activities of the components of the computing environment 800. The terms computing environment, computing node, computing system, and computer are used interchangeably.

The storage 840 can be removable or non-removable, and includes magnetic disks, magnetic tapes or cassettes, CD-ROMs, CD-RWs, DVDs, or any other medium which can be used to store information and that can be accessed within the computing environment 800. The storage 840 stores instructions for the software 880 and measurement data, which can implement technologies described herein.

The input device(s) 850 can be a touch input device, such as a keyboard, keypad, mouse, touch screen display, pen, or trackball, a voice input device, a scanning device, or another device, that provides input to the computing environment 800. The input device(s) 850 can also include interface hardware for connecting the computing environment to control and receive data from host and client computers, storage systems, or administrative consoles.

For audio, the input device(s) 850 can be a sound card or similar device that accepts audio input in analog or digital form, or a CD-ROM reader that provides audio samples to the computing environment 8Error! Reference source not found.00. The output device(s) 860 can be a display, printer, speaker, CD-writer, or another device that provides output from the computing environment Error! Reference source not found.00.

The communication connection(s) 870 enable communication over a communication medium (e.g., a connecting network) to another computing entity. The communication medium conveys information such as computer-executable instructions, compressed graphics information, video, or other data in a modulated data signal.

Some examples of the disclosed methods can be performed using computer-executable instructions implementing all or a portion of the disclosed technology in a computing cloud 890. For example, a primary filesystem can be in the computing cloud 890, while a disclosed file index can be operated in the computing environment.

Computer-readable media are any available media that can be accessed within a computing environment 800. By way of example, and not limitation, with the computing environment 800, computer-readable media include memory 820 and/or storage 840. As should be readily understood, the term computer-readable storage media includes the media for data storage such as memory 820 and storage 840, and not transmission media such as modulated data signals.

Any of the disclosed methods can be implemented using computer-executable instructions stored on one or more computer-readable media (e.g., non-transitory computer-readable media, such as one or more optical media discs, volatile memory components (such as DRAM or SRAM), or nonvolatile memory components (such as flash drives or hard drives)) and executed on a computer (e.g., any commercially available computer, proprietary computer, purpose-built computer, or supercomputer, including smart phones or other mobile devices that include computing hardware). Any of the computer-executable instructions for implementing the disclosed techniques, as well as any data created and used during implementation of the disclosed embodiments, can be stored on one or more computer-readable media (e.g., non-transitory computer-readable media). The computer-executable instructions can be part of, for example, a dedicated software application, or a software application that is accessed or downloaded via a web browser or other software application (such as a remote computing application). Such software can be executed, for example, on a single local computer (e.g., as a process executing on any suitable commercially available computer) or in a network environment (e.g., via the Internet, a wide-area network, a local-area network, a client-server network (such as a cloud computing network), or other such network) using one or more network computers.

For clarity, only certain selected aspects of the software-based implementations are described. Other details that are well known in the art are omitted. For example, it should be understood that the disclosed technology is not limited to any specific computer language or program. For instance, the disclosed technology can be implemented by software written in C, C++, Clojure, Common Lisp, Dylan, Erlang, Fortran, Go, Haskell, Java, Julia, Python, R, Scala, Scheme, SQL, XML, or any other suitable programming language. Likewise, the disclosed technology is not limited to any particular computer or type of hardware. Certain details of suitable computers and hardware are well-known and need not be set forth in detail in this disclosure.

Furthermore, any of the software-based embodiments (comprising, for example, computer-executable instructions for causing a computer to perform any of the disclosed methods) can be uploaded, downloaded, or remotely accessed through a suitable communication means. Such suitable communication means include, for example, the Internet, the World Wide Web, an intranet, software applications, cable (including fiber optic cable), magnetic communications, electromagnetic communications (including RF, microwave, and infrared communications), electronic communications, or other such communication means.

### VI. Representative Embodiments

Certain representative embodiments are exemplified in the following numbered clauses.

1. A device for plasma ligand capture, comprising: a body comprising a substrate material, wherein the body is an elongated body with a polygonal cross-section or wherein the body is an annular body; a poly(methyl methacrylate) (PMMA) coating on at least a portion of a surface of the body; and a plurality of capture moiety molecules covalently bound to the PMMA coating.

2. The device of clause 1, wherein the body is an elongated body with a polygonal cross-section, an upper surface, a lower surface, and a plurality of side surfaces, and the PMMA coating is on at least one of the side surfaces.

3. The device of clause 2, wherein the polygonal cross-section is cooperatively dimensioned to fit within a well of a 96-well plate or a neck of a vial or micro-centrifuge tube.

4. The device of clause 1, wherein the body is an annular body having an outwardly facing surface and an inwardly facing surface, and the PMMA coating is on at least a portion of the inwardly facing surface.

5. The device of clause 4, wherein the annular body has an outer diameter less than an inner diameter of a well of a 96-well plate or less than an inner diameter of a neck of a vial or micro-centrifuge tube.

6. The device of clause 5, further comprising an upper annular portion having an outer diameter greater than the inner diameter of the well or neck.

7. The device of any one of clauses 1-6, wherein the substrate material comprises a ferromagnetic metal, a polymer, or glass.

The device of any one of clauses 4-7, wherein the substrate comprises ferromagnetic steel.

9. A method, comprising: combining, in a vessel, a capture agent and a plasma sample comprising or suspected of comprising a ligand, the capture agent comprising biotin covalently attached to a protein capable of binding to the ligand; incubating the plasma sample and capture agent for a period of time effective for binding of the ligand, if present, to the capture agent to form a conjugate; inserting a device according to any one of clauses 1-8 into the vessel, whereby the conjugate binds to the capture moiety of the device; and removing the device with the bound conjugate from the plasma sample.

10. The method of clause 9, wherein the protein of the capture agent is a plasma protein.

11. The method of clause 9 or clause 10, wherein the plasma protein is human serum albumin (HSA), IgG, fibrinogen, transferrin, haptoglobin, α-1-acid glycoprotein (α-AGP), complement C, or a combination thereof.

12. The method of any one of clauses 9-11, further comprising: removing the conjugate from the device; combining the conjugate with a quantity of plasma or a solution comprising one or more proteins to provide an analysis sample, wherein the plasma or the solution comprising one or more proteins is devoid of the ligand; and obtaining a thermogram of the analysis sample by differential scanning calorimetry.

13. The method of clause 12, further comprising: inputting the thermogram into a computer system; comparing, using the computer system, the thermogram of the analysis sample to (i) a thermogram of a control sample comprising the plasma or the solution comprising one or more proteins, wherein the plasma or the solution is devoid of the ligand, (ii) a reference library of thermograms of samples comprising known ligands and plasma, samples comprising known ligands in solutions comprising one or more proteins, or both (i) and (ii) to provide a comparison; and determining, using the computer system and based at least in part on the comparison, whether the ligand is present in the analysis sample.

14. The method of clause 13, wherein the ligand is determined to be present in the analysis sample, the method further comprising, using the computer and based at least in part on the comparison, determining an identity, a quantity, or an identity and a quantity of the ligand in the analysis sample.

15. The method of any one of clauses 12-14, further comprising analyzing a portion of the conjugate removed from the device by chromatography, spectroscopy, gel electrophoresis, or a combination thereof to determine one or more properties of the ligand.

16. The method of any one of clauses 9-15, wherein the ligand is an exogenous compound or an endogenous component of the plasma.

17. The method of any one of clauses 9-16, wherein the ligand is a biomarker associated with a disease state or medical condition.

18. A method, comprising: obtaining a plasma sample of a subject, the plasma sample comprising or suspected of comprising a ligand; obtaining a thermogram of the plasma sample by differential scanning calorimetry; inputting the thermogram into a computer system; comparing, using the computer system, the thermogram of the plasma sample to (i) a thermogram of a control sample comprising plasma or a solution comprising one or more plasma proteins, the control sample being devoid of ligands, (ii) a reference library of thermograms of samples comprising known ligands in plasma or the solution comprising one or more plasma proteins, or both (i) and (ii) to provide a comparison; and determining, using the computer and based at least in part on the comparison, whether the ligand is present in the plasma sample.

19. The method of clause 18, wherein the ligand is determined to be present in the plasma sample, the method further comprising determining, using the computer and based at least in part on the comparison, an identity, a quantity, or an identity and a quantity of the ligand in the plasma sample.

20. The method of clause 19, further comprising diagnosing the subject with a disease or condition based at least in part on the identity, the quantity, or the identity and the quantity of the ligand in the plasma sample.

21. The method of clause 19, wherein the ligand is an exogenous therapeutic compound, the method further comprising determining a bioavailability of the exogenous therapeutic compound or a half-life of the exogenous therapeutic compound in the subject based on a quantity of the exogenous therapeutic compound in the plasma sample and an administered dosage of the exogenous therapeutic compound.

22. A method, comprising: combining a drug candidate with a quantity of plasma or a solution comprising one or more proteins to provide an analysis sample; obtaining a thermogram of the analysis sample by differential scanning calorimetry; inputting the analysis sample thermogram into a computer system; comparing, using the computer system, the analysis sample thermogram to a thermogram of a control sample comprising plasma or the solution comprising one or more proteins to provide a comparison, the control sample being devoid of the drug candidate; and determining, based at least in part on the comparison, whether the analysis sample thermogram exhibits a perturbation.

23. The method of clause 22, wherein the analysis sample thermogram exhibits a perturbation, the method further comprising: combining the drug candidate with a solution comprising one or more plasma proteins to provide a subsequent analysis sample; obtaining a thermogram of the subsequent analysis sample by differential scanning calorimetry; inputting the subsequent analysis sample thermogram into a computer system; comparing, using the computer system, the subsequent analysis sample thermogram to a thermogram of a control sample comprising the solution comprising one or more plasma proteins to provide a comparison; and determining, based at least in part on the comparison, whether the subsequent analysis sample thermogram exhibits a perturbation.

24. A non-transitory computer-readable medium storing instructions which, when executed by one or more processors, cause the processors to perform a method comprising: receiving an input sample record comprising a thermogram of a corresponding plasma sample and an identification of a ligand present in the plasma sample; and determining, using a trained machine learning model, a quantity or concentration of the ligand in the plasma sample.

25. The non-transitory computer-readable medium of clause 24, further comprising instructions which, when executed by the processors, cause the processors to incrementally grow the trained machine learning model using the determined quantity or concentration and at least part of the input sample record.

26. A method, comprising: establishing a feature vector specification derived from a thermogram specification, clinical history attribute specifications, and chemical and/or physical analysis output specifications; obtaining a plurality of labeled feature vectors, according to the feature vector specification, corresponding to respective samples; training a selected machine learning model with at least a portion of the plurality of labeled feature vectors; obtaining an unlabeled feature vector, according to a proper subset of the feature vector specification, corresponding to an unknown sample; and applying the trained machine learning model to the unlabeled feature vector to determine an identity or a quantity of a ligand present in the unknown sample.

27. The method of clause 26, wherein the obtaining the unlabeled feature vector comprises: receiving the unknown sample and clinical history data of the unknown sample; and performing a thermogram analysis on the unknown sample.

28. The method of clause 26, wherein the obtaining the unlabeled feature vector comprises: receiving a thermogram of the unknown sample and clinical history data of the unknown sample; and constructing the unlabeled feature vector from the thermogram and the clinical history data.

29. The method of any one of clauses 27-28, wherein the unknown sample or the thermogram of the unknown sample is received from a customer, and the method further comprises providing the determined identity or quantity of the ligand to the customer as a service.

30. The method of any one of clauses 26-28, further comprising, subsequent to the applying: determining that the trained machine learning model is inapplicable to a second sample; performing chemical and/or physical analysis on the second sample to obtain a second labeled feature vector, according to the feature vector specification, corresponding to the second sample; and incrementally growing the trained machine learning model using the second labeled feature vector.

31. A device for plasma ligand capture, comprising: a body comprising a substrate material, wherein the body is (i) an elongated body with a polygonal cross-section, or (ii) an annular body; a poly(methyl methacrylate) (PMMA) coating on at least a portion of a surface of the body; and a plurality of retrieval moiety molecules covalently bound to the PMMA coating.

32. The device of clause 31, wherein the body is an annular body having an outwardly facing surface and an inwardly facing surface, and the PMMA coating is on at least a portion of the inwardly facing surface.

33. The device of clause 32, wherein: (i) the annular body has an outer diameter less than an inner diameter of a well of a 96-well plate or less than an inner diameter of a neck of a vial or micro-centrifuge tube; or (ii) the annular body further comprises an upper annular portion having an outer diameter greater than an inner diameter of a well of a 96-well plate or less than an inner diameter of a neck of a vial or micro-centrifuge tube; or (iii) the substrate comprises ferromagnetic steel; or (iv) any combination of (i), (ii), and (iii).

34. The device of any one of clause 31-33, further comprising a capture moiety bound to at least one retrieval moiety molecule.

35. The device of clause 34, wherein: (i) the retrieval moiety molecule comprises streptavidin; or (ii) the capture moiety comprises biotin covalently attached to a protein capable of binding to a ligand of interest; or (iii) both (i) and (ii).

36. A method for retrieving a ligand from a plasma sample, comprising: combining, in a vessel, a capture moiety and a plasma sample comprising or suspected of comprising a ligand, the capture moiety comprising biotin covalently attached to a protein capable of binding to the ligand; incubating the plasma sample and capture moiety whereby the ligand, if present, binds to the capture moiety to form a conjugate; removing the conjugate, if present, from the plasma sample with a device according to any one of clauses 31-35.

37. The method of clause 36, wherein: (i) the ligand is an exogenous compound or an endogenous component of the plasma; or (ii) the ligand is an exogenous therapeutic compound.

38. The method of clause 36 or 37, wherein the protein of the capture moiety is a plasma protein, preferably wherein the plasma protein is human serum albumin (HSA), IgG, fibrinogen, transferrin, haptoglobin, α-1-acid glycoprotein (α-AGP), complement C, or a combination thereof.

39. The method of any one of clauses 36-38, wherein the device comprises the capture moiety, and combining the capture moiety and the plasma sample comprises inserting the device into the plasma sample.

40. The method of any one of clauses 36-39, further comprising: removing the ligand from the device; combining the removed ligand with a quantity of plasma or a solution comprising one or more proteins to provide an analysis sample, wherein the plasma or the solution comprising one or more proteins is devoid of the ligand; and obtaining a thermogram of the analysis sample by differential scanning calorimetry.

41. The method of clause 40, further comprising: inputting the thermogram into a computer system; comparing, using the computer system, the thermogram of the analysis sample to (i) a thermogram of a control sample comprising the plasma or the solution comprising one or more proteins, wherein the plasma or the solution is devoid of the ligand, (ii) a reference library of thermograms of samples comprising known ligands and plasma, samples comprising known ligands in solutions comprising one or more proteins, or both (i) and (ii) to provide a comparison; and determining, using the computer system and based at least in part on the comparison, whether the ligand is present in the analysis sample.

42. The method of clause 41, wherein the ligand is determined to be present in the analysis sample, the method further comprising: (i) using the computer and based at least in part on the comparison, determining an identity, a quantity, or an identity and a quantity of the ligand in the analysis sample; or (ii) analyzing a portion of the ligand removed from the device by chromatography, spectroscopy, gel electrophoresis, or a combination thereof to determine one or more properties of the ligand; or (iii) both (i) and (ii).

43. The method of clause 42, wherein the plasma sample is obtained from a subject, the method further comprising diagnosing the subject with a disease or condition based at least in part on the identity, the quantity, or the identity and the quantity of the ligand in the plasma sample.

44. The method of clause 42, wherein the plasma sample is obtained from a subject and the ligand comprises an exogenous therapeutic compound, the method further comprising: comparing, using the computer system, the thermogram of the plasma sample to (i) a thermogram of a control sample comprising plasma or a solution comprising one or more plasma proteins, the control sample being devoid of the exogenous therapeutic compound, (ii) a reference library of thermograms of samples comprising the exogenous therapeutic compound in plasma or the solution comprising one or more plasma proteins, or both (i) and (ii) to provide a comparison; determining, using the computer and based at least in part on the comparison, presence of the exogenous therapeutic compound in the plasma sample; determining, using the computer and based at least in part on the comparison, a quantity of the exogenous therapeutic compound in the plasma sample; and determining a bioavailability of the exogenous therapeutic compound or a half-life of the exogenous therapeutic compound in the subject based on a quantity of the exogenous therapeutic compound in the plasma sample and an administered dosage of the exogenous therapeutic compound.

45. A method for drug discovery or analysis, comprising: (a) combining a quantity of a drug candidate with a quantity of a solution comprising one or more plasma proteins to provide an analysis sample; (b) obtaining a thermogram of the analysis sample by differential scanning calorimetry; (c) inputting the analysis sample thermogram into a computer system; (d) comparing, using the computer system, the analysis sample thermogram to a thermogram of a control sample comprising the solution comprising one or more plasma proteins to provide a comparison, the control sample being devoid of the drug candidate; (e) determining, based at least in part on the comparison, whether the analysis sample thermogram exhibits a perturbation; and (f) if a perturbation is exhibited, (i) repeating steps (a)-(e) with one or more additional quantities of the drug candidate; and (ii) determining, based at least in part on the perturbation, a characteristic of an interaction of the drug candidate with the one or more plasma proteins, wherein the characteristic is a binding constant, reaction enthalpy, binding stoichiometry, binding free energy, binding entropy, or any combination thereof.

46. The method of clause 45, wherein the drug candidate has an aqueous solubility ≤10 mg/ml and combining the quantity of the drug candidate with the quantity of the solution comprising one or more plasma proteins further comprises:

providing a solution comprising the quantity of the drug candidate and an organic solvent;

evaporating the organic solvent from the solution to provide the quantity of the drug candidate in solid form; and

combining the quantity of the drug candidate in solid form with the quantity of the solution comprising the one or more plasma proteins.

### VII. Examples

### Methods:

Chemicals and reagents: Plasma and highly pure plasma proteins are sourced from commercial suppliers. Standard reagents are sourced from commercial suppliers. Samples are prepared in standard phosphate-buffered saline (PBS) buffer. In some examples, human plasma and serum albumin were purchased from Sigma Aldrich (St. Louis, Mo.) and received as lyophilized powder. Plasma was product number: P9523, lot number: SLBT0202. Human serum albumin (HSA) advertised as fatty acid and globulin free, ≥99% pure was lot number: SLBD7204V. This definition of “standard” HSA is strictly applicable in an in vitro system where fatty acids have been removed. In vivo, the standard state of HSA is most certainly bound to some extent by fatty acids. Plasma and HSA stock solutions were prepared by re-suspending the appropriate amount of powder in buffer. Samples were prepared by diluting stock solutions to a final concentration of 1.5-2.0 mg/mL.

Instrumentation: Differential scanning calorimeters are Nano II DSC (TA Instruments, Wilmington, Del.). Thermo Scientific LTQ-Orbitrap® Discovery mass spectrometer (San Jose, Calif.) with electrospray ionization source. Fluorimeter is a Haribo PTI Quantamaster 3000 (Kyoto, Japan). UV-visible spectrophotometer is an Agilent® 8453 spectrophotometer (Santa Clara, Calif.).

Determination of protein concentrations: Protein concentrations were determined as previously described (Hoang et al., J Biophys Chem 2016, 7(01):9) using the BCA method and the Protein Assay Kit (product #23225, Thermal Fisher Scientific).

Ligand samples: Ligands included: (1) naproxen (NAP); (2) bromocresol green (BCG) and (3) short single strand (ssDNA) and double stranded DNAs (dsDNA). BCG product number: 114359, lot number: 07896HJ; and NAP product number: N8280, lot number: 040M1400V were purchased from Sigma Aldrich (St. Louis, Mo.). The 25-base pair double stranded (25-mer) and the individual strands (25R and 25L) that comprise it were purchased from IDT and received after having been subjected to their standard desalting routine. The 25R DNA sequence is 5′-CGA CAT GAC CTT GTC GCT AAC ATC C-3′ (Ref. No. 165820905) DNA 25L is the perfect complement of DNA 25 R; and DNA 25-MER, the 25-base pair duplex made from 25R+25L. 25R with a 5′ cy-5 fluorescent label was also purchased from IDT and received as HPLC purified and desalted. Labeled 25-MER was prepared by incubating 5′ cy-5 labeled 25R with its complement, 25L. To ensure all duplex molecules were labeled, the two strands were mixed with a slight excess of unlabeled strand in a 1:1.01 molar ratio. The mixture was heated to 90° in a heat block, the heater was turned off and the sample was allowed to slowly cool back to room temperature.

Ligand solubilization: Stock solutions of aqueous insoluble ligands in organic solvents were pipetted into microcentrifuge tubes to yield a desired amount of drug for a 1 mL solution. The microcentrifuge tubes were placed into a vacuum concentrator (Savant SpeedVac Model SC100), and the organic solvents were evaporated, resulting in solid drug in the tube. To each tube, HSA is added at a predetermined concentration with standard buffer to provide a 1 mL working solution. HSA is known is to allow plasma concentrations of ligands to exist in concentrations above the solubility limit in aqueous solution.

Solvents and reagents: Standard PBS buffer solutions contained 10 mM potassium phosphate and 150 mM NaCl, pH=7.4. Total ionic concentrations of buffers were verified by electrical conductivity measurements. After preparation and prior to use buffer solutions were stored at 4° C. TBST magnetic bead wash buffer was 20 mM Tris-HCl, 150 mM NaCl and 0.1% Tween20, pH=7.5. Retrieval high-salt wash buffer was 20 mM Tris-HCl and 500 mM NaCl, pH=7.5. Isolation low-salt wash buffer was 20 mM Tris-HCl, pH=7.5. SDS: 5% Sodium Dodecyl Sulphate, 50% glycerol and 12.5 mM Tris-HCl, pH=8.0. Retrieval wash solution, 50:0.1:49.9 (v/v %) acetonitrile:acetic acid:H₂O. All solutions and buffers were prepared with nanopure deionized water. Chemicals and reagents were molecular biology grade or higher.

Gel electrophoresis staining: Stains-All was purchased from Sigma Aldrich (St. Louis, Mo.) (product number: E9379, lot number: BCBS0570V). Staining solution was 60% 20 mM Tris-HCl pH=8, 20% isopropanol, 20% 0.1% Stains-All in formamide.

Gel electrophoresis: DNA samples collected at different steps of the capture procedure were analyzed by electrophoresis on polyacrylamide gels. All electrophoresis experiments were performed using Lonza PAGEr™ Gold Precast Gels: Gradient, 10×10 cm, 8-16%, purchased from Thermo-Fisher (BMA59519). Each supernatant fraction collected at different steps of the capture procedure were suspended in TAE running buffer and analyzed. In a typical experiment, 25 μL total volume of solution was loaded per lane (well capacity). Gels were run in TAE buffer (40 mM Tris, 20 mM Acetic Acid, 0.4 mM EDTA) at a constant current of 20 mA for approximately three hours. Gels for analysis of DNA were stained with Stains-all solution, destained in water, removed, visualized and imaged on a flat-bed scanner. Gels for analysis of hot labeled cy5′-25MER DNA were visualized using a Typhoon™ Trio+phosphorimager (GE Healthcare).

High-pressure liquid chromatography (LC) and mass spectroscopy (MS): Samples were analyzed for the presence of analyte (ligand) using HPLC-MS instrumentation consisting of an Accela HPLC system (Thermo Fisher Scientific) coupled to an electrospray ionization source and LTQ-Orbitrap Discovery high resolution mass spectrometer (ThermoElectron). Retrieved analyte samples were separated using a 50 mm BetaBasic 18 HPLC column (internal diameter 1 mm; C18 3 μm; Thermo Fisher Scientific). Each LC-MS analysis used 10 μL of sample with a run time of 10 minutes. Ligand samples were kept in the retrieval wash solution and loaded in buffer A (0.1% (v/v %) formic acid) and eluted using a linear 5 minute gradient (5-95% buffer B comprised of 0.1% (v/v %) acetic acid, 99.9% (v/v %) acetonitrile) held for 2 minutes at 95% (v/v %) buffer B, followed by a 3 minute wash of 95% (v/v %) buffer A, 5% (v/v %) buffer B. All flow rates were held constant at 500 μL/min and the column temperature was maintained at 35° C. MS data was acquired using the combination of a low-resolution ion trap and high resolution FTMS. Targeted values for detection of the ligands was set for a scan range of 100.00-750.00 m/z and a resolution of 30,000 at m/z=400. Samples were ionized in negative mode with a spray voltage of 2.50 kV and normalized collision energy of 35.0 eV.

MS data analysis: MS raw data files were analyzed using Xcalibur software version 4.1 (Thermo Scientific). MS data are displayed in standard form as plots of relative abundance versus the m/z ratio. Isotope simulation of mass spectra identified target ligands with an allowed mass deviation of less than 20 ppm.

Differential scanning calorimetry: DSC melting experiments were performed using a CSC Model 6100 Nano II-Differential Scanning calorimeter (formerly calorimetry Sciences Corporation, Provo Utah, now TA Instruments). The average of three to five buffer scans collected over the temperature range from 0 to 100° C. served as the buffer baseline for analyzing scans of protein, ligand samples, and their mixtures. A temperature scan rate of 1° C./min was employed. Protein concentration was approximately 2 mg/ml. The temperature range used for measuring DSC thermograms was typically from 25 to 90° C. For displayed thermograms, the range was 45 to 90° C. and all raw data was smoothed using a non-parametric local regression (LOESS) method.

In a DSC melting experiment, the supplemental power supplied to the sample cell (in μW) necessary to keep the sample temperature equal to the reference temperature is continually monitored. Simultaneously, temperatures of the sample and reference cells are linearly increased at precisely the same rate. Supplemental power is directly related to the molar heat capacity at constant pressure, ΔCp. Curves of ΔCp versus T provide an evaluation of the thermodynamic enthalpy. To enable direct comparisons in some cases, power (μW) versus T plots (instead of ΔCp versus T curves) were used for the following reason.

In the standard analysis of solutions containing a single molecular species conversion of the raw signal in μW to ΔCp values requires precise knowledge and input of the sample mass/mL, cell volume, MW, and partial specific volume (PSV). For a mixture of molecules of different types, as is the case for HSA+DNA, where both components of the mixture have appreciable ΔCp values with some overlap over the same temperature range, application of the standard analysis, based on the presence of a single type of molecular species, precludes proper comparison. That is, composite melting curves of the mixtures can only be analyzed in the standard way assuming a single MW, i.e. either 66 kD or 3-5 kD for both HSA and DNA, and a single PSV for both, which is patently incorrect! To circumvent this limitation for comparison purposes, thermograms for DNA/HSA_{B} (biotinylated HSA) and DNA/plasma mixtures were constructed by plotting total power in μW versus T. These curves were normalized and compared to μW versus T to thermograms measured for HSA, HSA_{B}, or plasma alone at precisely the same concentrations as in the mixtures. Using normalized curves constructed from μW (instead of ΔCp versus T) provides a valid means for comparison, but also introduces a limitation on quantitative information obtained. That is, in order to establish an appropriate footing for comparison of composite melting curves of DNA/plasma and DNA/HSA_{B} mixtures, we lose the ability to quantitatively evaluate the molar thermodynamic enthalpies of the mixtures

Analysis of DSC data was performed using the Nanoanalyze software package, version 3.7.5, provided by T.A. Instruments. Steps in the analysis procedure for analysis of μW versus T curves were precisely the same as reported for ΔCp versus T data.25 For analysis of thermograms of the ligands (NAP, BCG, and DNA), HSA, biotinylated HSA (HSA_{B}), and plasma alone, the standard analysis procedure was employed exactly as described previously.25 Values of the calorimetric transition enthalpy, ΔH_{cal}, determined from the integrated area under the measured thermogram ΔCp (T) versus T curves were used to asses quality of HSA (and HSA_{B}) samples and characterize HSA/ligand complexes.

Analytical ultracentrifugation: Sample preparations of biotinylated HSA_{B} containing different levels of HSA:biotin attachment at ratios of 1:1, 1:5 and 1:10 were characterized by AUC. The procedure provides a highly accurate measurement of HSA dimer/monomer populations and MW of the different biotinylation levels and reveals significant changes in structure and conformation (if they exist) with increased biotinylation.

Biotinylation: In some examples, the capture moiety is biotinylated HSA (HSA_{B}) made using the N-hydroxy succinate biotin (N-HS) reagent (product number 21217 from Thermo Fisher Scientific) as described in Hoang et al. (J. Biophys. Chem. 2016, 7(01):9), which attaches biotin to primary amines of lysine resides. Standard buffer for all experiments contained 150 mM NaCl, 10 mM potassium phosphate, 15 mM sodium citrate, pH=7.4. Capture experiments for capture moieties prepared at incubation ratios (biotin:HSA) of approximately 10:1, 5:1, and 1:1, suggested the intermediate coverage produced the best capture (not shown). For the capture moiety used in this study, HSA_{B} was prepared at an estimated coverage of 5:1. Based on DSC measurements, biotinylation does not greatly perturb overall structural stability of the protein. AUC measurements concur. MW determinations by AUC were found to be accurate to within +/−5 kDa (Zhao et al., PLoS One 2015, 10(5):e0126420). Thus, an increase of 2.443 kDa (corresponding to attachment of 10 biotins) HSA_{B} would have a MW within the error of the measurement. AUC measurements indicated for HSA_{B} at a 1:10 HSA:biotin attachment ratio a MW of 56.7 kDa; at a 1:5 attachment ratio MW of 64.6 kD and at a 1:1 attachment ratio MW=63.3 kD. These MW values are essentially the same within the error of AUC measurements for unmodified natural HSA. For the biotinylated species monomer/dimer ratios were approximately 90% monomer, 10% dimer indicating no change in dimerization dissociation constant with increased biotinylation. Monomer frictional ratios were also quite similar indicating no differences in shape. Overall, results of AUC analysis were consistent with DSC measurements; and also indicated biotinylation of HSA does not alter gross conformation, stability, or binding capacity of the protein.

Capture strategy: In some examples, biotinylated HSA acts as an affinity reagent for ligands in plasma that bind HSA. In the capture step, streptavidin coated magnetic beads are attached to biotinylated HSA then inserted into plasma. With application of a magnetic field, ligand-bound biotinylated HSA is retrieved. Captured HSA contains bound plasma components (ligands).

Bound ligands are washed off the retrieved biotinylated HSA and subjected to further characterization and analysis by gel electrophoresis and MS. In certain examples, the retrieval moiety is a magnetic bead, surface-coated with streptavidin. Coupling of the capture and retrieval moieties is achieved through the biotin-streptavidin linkage, resulting in the fully complete capture reagent. Coupled reagents are separated from uncoupled reactants using a magnet while pulling off the supernatant; the magnet is removed, and the retained coupled capture reagent is re-suspended in appropriate buffer.

Ligand recovery: In the capture process, HSA-bound components are washed off the capture reagent. The wash protocol employs a mixture of weak acid and organic solvent for small molecule drugs. For DNA, high salt washes were used. With this combination of solvents, the HSA-bound components are presumably washed off the capture reagent. Given the milieu of plasma molecular components and ligands such as proteins, peptide fragments, nucleic acids, fatty acids and lipids that can potentially be bound, it is not surprising diverse solvent washes might be required to dislodge bound components of various types.

### Example 1

### Device Manufacture

In some examples, base substrate, such as a ferromagnetic material, was coated with a solution comprising PMMA. In some examples, coating was done with a spin coater. The coated surface was heated at 200° C. to evaporate solvent from the PMMA solution. After drying, the polymer surface is hardened and shelf stable. The coated metal was then punched or cut into the desired shape. To enable capture moiety attachment, surface PMMA was functionalized by exposure to O₂ plasma to create carboxylic acid groups. In particular, the capture moiety (streptavidin) was attached using 1-ethyl-3-(3-dimetylaminopropyl) carbodiimide and 2-(N-morpholino)ethanesulfonic acid) buffer (Vesel et al., Vacuum 2012, 86(6):773-775). The capture agent, biotinylated HSA, was prepared as described above. The capture agent was attached to the capture moiety-coated capture device by incubating the device in solution with the capture agent.

In other examples, biotin was attached to HSA using the EZ-Link Sulfo-NHS-Biotin kit (product number 21217 from Thermo Fisher Scientific) according to the supplier's instructions. For attachment reactions, a 10 mM stock solution of Biotin was prepared by dissolving Biotin in water. A solution containing a 1:5 molar ratio of HSA:Biotin was prepared by adding appropriate amounts of the Biotin stock solution to an HSA solution at 2 mg/mL, and was stored at 4° C. for at least 24 hours. When attachment reactions were complete, free (unattached) Biotin was removed using a Zeba™ spin desalting column (product number: 89892, lot number: RH236113A, Thermo Scientific). In this procedure, the column was equilibrated three times with 2 mL standard PBS buffer. An aliquot of 1.5 mL of the attachment reaction solution was then added directly to a spin column and retrieved. Sample volumes were such that several columns were required. Retrieved products from these runs were pooled. Pierce™ streptavidin magnetic beads were purchased from Thermo Scientific (product number: 88816, lot number: SG249234). Magnetic beads were prepared according to the supplier's instructions, by rinsing 50 μL (0.5 mg) of beads with 1 mL TBST wash buffer. After removal of the wash buffer, 300 μL of the capture moiety was added to the beads and the mixture was incubated at 4° C. for at least 24 hours. After incubation, the sample was placed under a magnetic field and excess capture moiety in the supernatant was removed. Remaining capture reagent was never allowed to completely dry and was stored in buffer for future use.

### Example 2

### General Process for Ligand Capture and Analysis

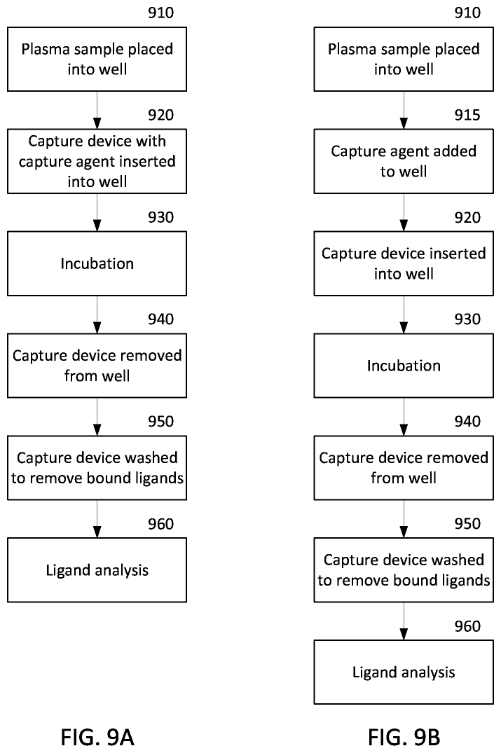

FIG. 9A is a flowchart showing one embodiment of a method for capturing and analyzing a ligand from a plasma sample. Plasma samples are placed into a well of a 96-well microplate or into a vial (910). A capture device comprising a capture agent is inserted into the well (920) and incubated for a period time sufficient to allow binding of at least some ligands in the plasma sample to the capture agent (930). The capture agent typically is a biotinylated protein capable of binding to the ligand. The capture device is removed manually or via magnetic transfer from the well (940) for further analysis. Plasma remaining in the well may be subjected to diagnostic tests and/or subjected to DSC to provide a plasma thermogram. The capture device is washed to remove bound ligands (950). In some embodiments, the wash comprises an acidified alkanol/water solution, such as acidified ethanol/water. In some examples, the capture device is washed with 50% (v/v) ethanol, 0.1% (v/v) acetic acid. The removed ligands are further analyzed (960), e.g., by liquid chromatography/mass spectroscopy, DSC, or other techniques.

FIG. 9B is a flowchart showing another embodiment of a method for capturing and analyzing a ligand from a plasma sample. The process of FIG. 9B differs from that of FIG. 9A in that the capture device does not comprise the capture agent. Instead, the capture agent is added to the well (915), and the capture device is subsequently added (920). During the washing step (950), the ligand is removed from the capture device, but the biotinylated capture agent remains bound to the capture moiety of the capture device.

### Example 3

### Capture of Naproxen and Bromocresol Green

A capture device comprising biotinylated human serum albumin (HSA) as the capture agent was used to capture naproxen and bromocresol green from human plasma samples as described in Example 2. NAP binds with n=1 to Sudlow site II of HSA, which contains a hydrophobic pocket. BCG is a Sudlow site I binder with n=3.

NAP and BCG (Sigma Aldrich) were used as received from the supplier without further purification. For these reactions 1 mL solutions containing 1 mg/mL human plasma or 2 mg/mL HSA and 100 μM of either NAP or BCG were incubated at 4° C. for 24 hours. The capture reagent (biotinylated HSA coupled to streptavidin-coated magnetic beads) was added to the plasma solutions and incubated for 24 hours at 4° C. A magnetic field was applied the supernatant removed. Before retrieval of the ligands, beads were washed with 300 μL of 0.1% Tween® 20 surfactant. The retrieval wash contained acetonitrile, acetic acid, and water in a ratio of 50:0.1:49.9 (v/v %) with 150 mM NaCl at pH 3.5. The capture reagent was washed with 100 μL retrieval wash solution and vortexed for 10 seconds. This was repeated, and aliquots were combined for a total volume of 200 μL. Using mass spectroscopy, it was estimated from the ion count that the NAP concentration in the retrieval wash was approximately 2 μM. Similar to NAP, the concentration of BCG in the wash solution was estimated to be 2-3 μM.

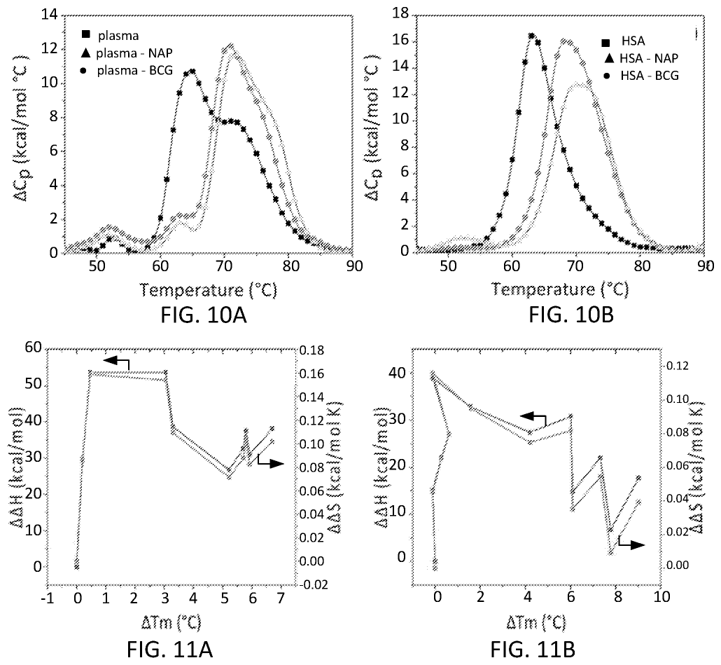

FIGS. 10A and 10B are thermograms showing the effects of 100 μM naproxen (NAP) and 100 μM bromocresol green (BCG) on plasma (10A) and HSA (10B). In both plasma and a standard solution of HSA, 100 μM NAP produces a shift in Tm of about 5° C., and 100 μM BCG shifts Tm about 7° C. For 100 μM NAP, ΔH_{cal} was 181.7 kcal/mol, compared to 155.0 kcal/mol for HSA alone. For 100 μM BCG, ΔH_{cal} was 165.6 kcal/mol for the HSA/ligand and only slightly higher than HSA alone.

As shown in FIG. 10A, NAP interacts with HSA in plasma, affecting the thermogram. Plasma has three Tm peaks at 53° C., 64° C., and 71° C. representing the major plasma proteins fibrinogen, HSA, and globulins respectively, and a ΔH_{cal} of 177 kcal/mol. when NAP interacts with plasma, the major Tm peak of 63° C. is shifted to 70° C. and the enthalpy decrease to 149 kcal/mol. This is confirmed in FIG. 10B where NAP with HSA shows a similar shift in Tm and an increase of ΔH_{cal} from HSA (154 kcal/mol) to 181 kcal/mol. Similar to NAP, when BCG interacts with plasma the major Tm peak is shifted to 72° C. and the enthalpy decreases to 141 kcal/mol (FIG. 10A). This interaction can also be attributed to BCG interaction with HSA. In FIG. 10B it is shown that BCG with HSA has a similar shift in Tm (˜7° C.) and a minor increase in ΔH_{cal} to 166 kcal/mol.

Concentrations of NAP and BCG were varied, and semi-quantitative evaluations of thermodynamic quantities, ΔH_{cal} and ΔS_{cal} (≈ΔH_{cal}/Tₘ), were made at the various ligand concentrations. For both NAP and BCG, the Tm remained constant at low concentrations (e.g., ≤50 μM), and then increased incrementally, in a generally linear manner, through higher concentrations. The results are plotted in FIGS. 11A and 11B, where the differences ΔΔH_{cal} and ΔΔS_{cal} between parameters evaluated from thermograms for ligand/HSA mixtures and those for standard HSA alone are plotted versus differences in transition temperatures, ΔTm, between thermograms for the ligand mixtures, at increasing ligand concentrations and standard HSA. The figures show differences in the modes of binding HSA for the two ligands. At lower ΔTm (ligand concentration), values in the curves are similar and display a rapid rise at the lowest concentrations, with a leveling off at intermediate concentrations. At the higher ligand concentrations, behaviors are different. For NAP (FIG. 11A), after a slight decrease, the values are essentially constant at higher concentrations. In contrast, with BCG (FIG. 11B), after the initial increase, values consistently trend lower over the remainder of the concentration range. These differences are indicative of the different binding stoichiometries for NAP (n=1) and BCG (n=3) for their binding sites on HSA. The curves in FIG. 11A suggest saturation, while those in FIG. 11B do not, entirely consistent with the different binding capacities for the two ligands. Further evidence that NAP and BCG were effectively captured was demonstrated by mass spectroscopy and a blue color of the retrieved ligand solution corresponding to BCG.

From the thermograms, semi-quantitative evaluations of the thermodynamic parameters ΔH_{cal}, ΔS_{cal} and ΔG_{cal}(37° C.) were obtained at each ligand concentration. These evaluated thermodynamic parameters for thermal denaturation of HSA bound by NAP and BCG are summarized in Table 1. Standard error on ΔH_{cal} and ΔS_{cal} values was approximately 5%. ΔG_{cal}(37° C.) values were within ±0.2 kcal/mol and Tₘ's were ±0.15° C. Examination of these results revealed significant differences for the two ligands as a function of concentration.

TABLE 1
Thermodynamic Parameters of Binding

| Naproxen | | | | | Bromocresol green | | | | |
|---|---|---|---|---|---|---|---|---|---|
| [NAP] | ΔH_{cal} | ΔS_{cal} | ΔG₃₇^{O} | Tₘ | [BCG] | ΔH_{cal} | ΔS_{cal} | ΔG₃₇^{O} | Tₘ |
| (μM) | (kcal/mol) | (cal/molK) | (kcal/mol) | (° C.) | (μM) | (kcal/mol) | (cal/molK) | (kcal/mol) | (° C.) |
| 0 | 154.94 | 460.93 | 11.98 | 62.5 | 0 | 154.94 | 460.93 | 11.98 | 62.5 |
| 1 | 184.38 | 548.18 | 14.36 | 63.2 | 1 | 170.12 | 506.23 | 13.11 | 62.9 |
| 10 | 208.65 | 619.86 | 16.40 | 63.5 | 10 | 176.99 | 526.08 | 13.83 | 63.3 |
| 25 | 211.07 | 625.67 | 17.02 | 64.2 | 25 | 182.05 | 540.59 | 14.39 | 63.6 |
| 50 | 208.56 | 614.86 | 17.86 | 66.1 | 50 | 189.75 | 562.81 | 15.19 | 64.0 |
| 75 | 204.50 | 601.63 | 17.90 | 66.8 | 75 | 187.76 | 555.95 | 15.33 | 64.6 |
| 100 | 192.45 | 563.76 | 17.60 | 68.2 | 125 | 182.33 | 535.71 | 16.18 | 67.2 |
| 150 | 193.64 | 566.45 | 17.96 | 68.7 | 150 | 178.84 | 522.66 | 16.74 | 69.0 |
| 175 | 192.52 | 563.00 | 17.90 | 68.8 | 175 | 169.77 | 496.04 | 15.92 | 69.1 |
| 200 | 192.04 | 561.41 | 17.92 | 68.9 | 200 | 167.42 | 487.44 | 16.24 | 70.3 |
| | | | | | 22.5 | 167.24 | 486.25 | 16.43 | 70.8 |
| Error | 7% | 7% | ±0.2 | ±0.2 | Error | 7% | 7% | ±0.2 | ±0.2 |

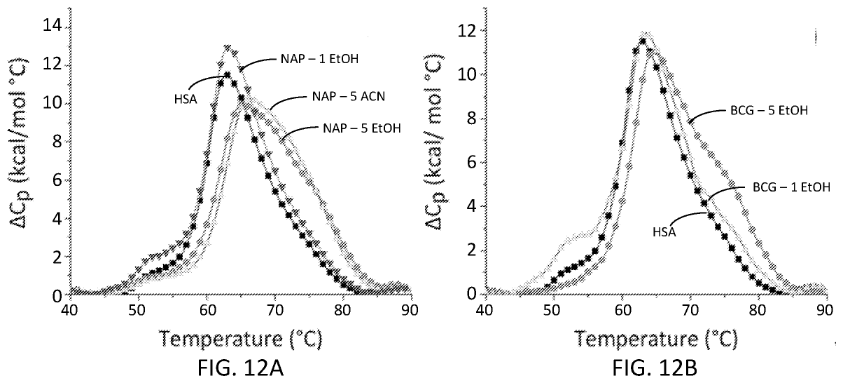

Capture and retrieval of NAP and BCG were performed again, with the capture device sequentially inserted into the plasma and washed five times. The washings were combined and concentrated by removing solvent using a Speed-vac concentrator. The dried solids were suspended in a 2 mg/mL standard HSA solution. NAP was recaptured under three different conditions: (1) NAP washed with acidified acetonitrile repeated 5 times totaling 1 mL; (2) NAP washed with acidified ethanol repeated 5 times totaling 1 mL; and (3) NAP washed a single time with acidified ethanol. Each was allowed to incubate in 500 μL of 2 mg/mL standard HSA. Recaptured solutions were analyzed using DSC and compared to the standard HSA thermogram (FIG. 12A). The recaptured solutions pooled from ethanol and acetonitrile were extremely similar and showed a Tm shift of ˜4° C.; compared to the results of FIG. 11A, a similar Tm shift was seen for concentrations of NAP between 50 and 100 μM. Recapture of a single concentrated analyte resulted in an increase in ΔH and no noticeable Tm shift, analogous to NAP concentrations of 1-10 μM. By shifting the Tm of HSA, it can be concluded that NAP was responsible for perturbation of the plasma thermogram and was caused by NAP binding to HSA.

BCG was recaptured with using 1 or 5 acidified ethanol washes. As shown in FIG. 12B, clear differences are seen between the HSA thermogram and the thermograms with recaptured BCG. The single wash resulted in a thermogram only slightly perturbed from HSA. At low concentrations, ≤10 μM, BCG has very little effect on the thermogram. At 1 μM, there was no apparent change in the thermogram (not shown). This suggests that the recovered concentration of BCG was around 10 μM. The thermogram from the pooled ethanol washes shows a characteristic shape for BCG—a slight Tm shift with appearance of a secondary peak at −70° C. Concentrations of BCG between 50 and 75 μM show this characteristic thermogram.

The recapture process and subsequent analysis demonstrated that ligands retrieved from plasma can be verified according to their perturbation of a standard HSA thermogram. Preliminary results showed that thermograms of the add-back mixtures provided enough detail to confirm their presence in the captured products. As demonstrated, thermograms of recaptured ligand+HSA mixtures were noticeably different from standard HSA and from one another. Such differences in the HSA thermograms can be used to positively differentiate ligands whose effects on the standard plasma thermogram, although perturbed, are very similar.

### Example 4

### Relational Database Development and Use

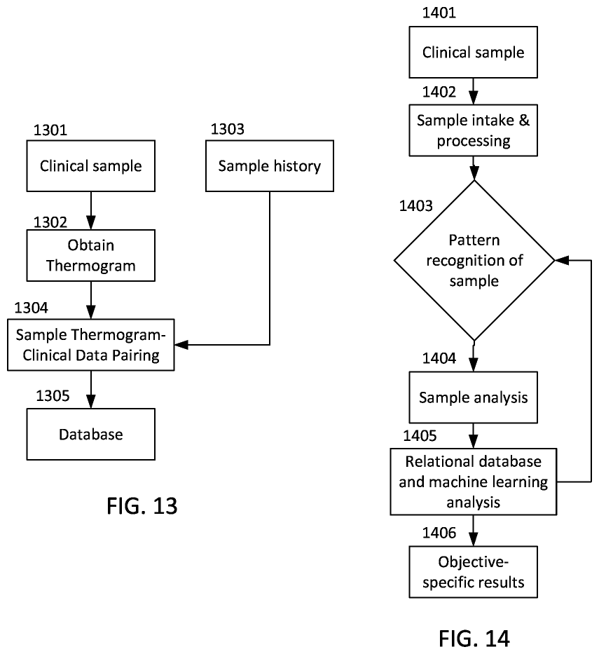

A generalized process for building a thermogram database is shown in FIG. 13. A clinical plasma sample is obtained from a patient or prepared using analyte standards (1301). A thermogram of the sample is obtained (1302). Sample history (e.g., drug identification, patient status, etc.) is obtained (1303) and paired with the sample and the thermogram (1304). The paired data is transmitted to a computer database, such as a relational database (1305).

A generalized process for drug development, therapeutic monitoring, and patient health status monitoring is shown in FIG. 14. A clinical plasma sample is obtained from a patient or prepared using analyte standards (1401). Sample history (e.g., drug identification, patient status, etc.) is paired with the sample, e.g., by inputting the sample history into a computer database and a thermogram of the sample is obtained (1402). A machine learning algorithm as disclosed herein is used to identify and flag samples for further testing (1403). Samples are then analyzed (1404), and the analysis results are transmitted to a relational database, thereby enhancing the details and capabilities of the machine learning algorithm and providing detailed analysis (1405). Results of the pattern recognition and analysis provides objective-specific results for the user (1406).

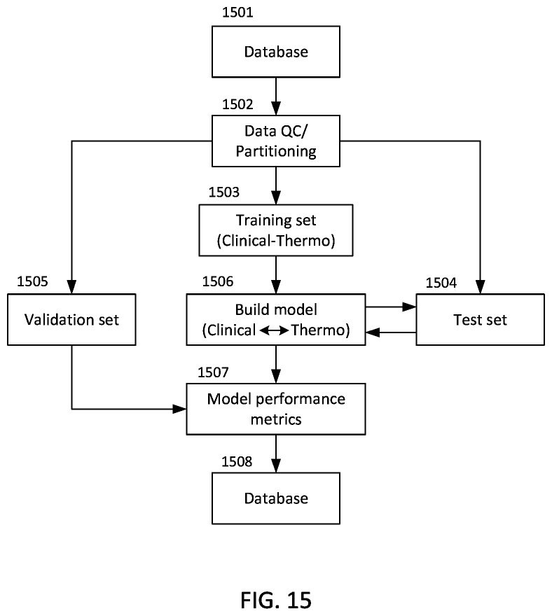

FIG. 15 illustrates an exemplary process for a machine-learning model. A database 1501 including thermograms and clinical sample data is provided. A data quality control and partitioning process 1502 is performed, and data is assigned to a training set of clinical and thermogram data 1503, a test set 1504 and/or a validation set 1505. The training set 1503 is used to build a model 1506 including both clinical and thermogram data. Interactions between the test set 1504 and model 1506 are used to further develop the model. The validation set 1505 is utilized to determine model performance metrics 1507. The model performance metrics 1507 are included in the database 1508.

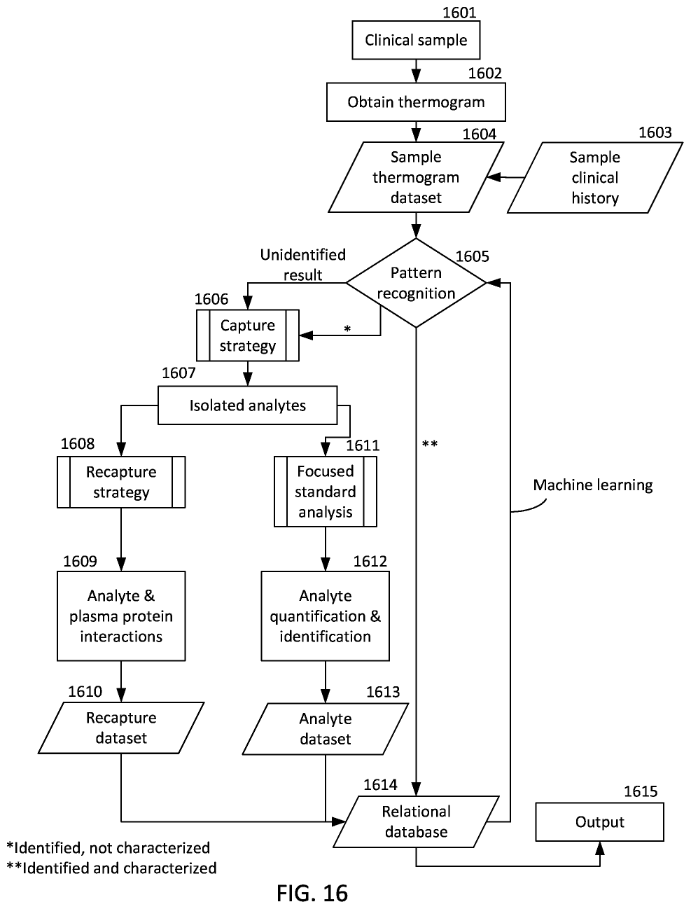

An exemplary process for developing a relational database is illustrated in FIG. 16. As one nonlimiting example, a clinical sample includes naproxen (NAP). Initially, the clinical sample is procured (1601) and a thermogram is obtained (1602). The sample clinical history is obtained (1603). The sample clinical history and thermogram are inputted into a sample thermogram dataset (1604). The thermogram is compared to a thermogram for standard plasma (1605). The sample thermogram is determined to statistically different from the standard plasma thermogram (see, e.g., FIG. 10A for thermograms of standard plasma and plasma including NAP). If the analyte is unidentified, the clinical sample is subjected to the capture strategy (1606) (e.g., as described in FIG. 14). Analytes are isolated form the clinical sample (1607). The isolated analytes are subjected to a recapture strategy (1608) and/or focused standard analysis (1611). The recapture strategy elucidates an analyte's interactions with plasma proteins, which cause the thermogram perturbation (1608). Differential scanning calorimetry and analyte titrations are used to characterize the analyte's influence on particular plasma proteins, e.g., albumin (1609) (see, e.g., FIG. 10B for thermograms of HSA and HSA with NAP). Thermograms of the analyte's effect on individual plasma proteins are stored in the recapture dataset (1610). Isolated analytes also are subjected to standard analytical chemistry techniques, such as NMR, chromatography, mass spectroscopy, and the like (1611). Results of the focused standard analysis provide a positive identification of a known analyte/ligand, or can be used to identify an unknown ligand, and provide quantitative data (1612). The analysis results are stored in an analyte dataset (1613). Results stored in the sample thermogram, recapture, and analyte databases are linked in the relational database to build a profile for the sample and analyte (1614). An output of the profile may be obtained (1615). Subsequent samples continue to feed into the relational database, increasing the amount of data contained in an analyte profile. A machine learning algorithm is used to parse the data and enhance the pattern recognition capabilities of the system.

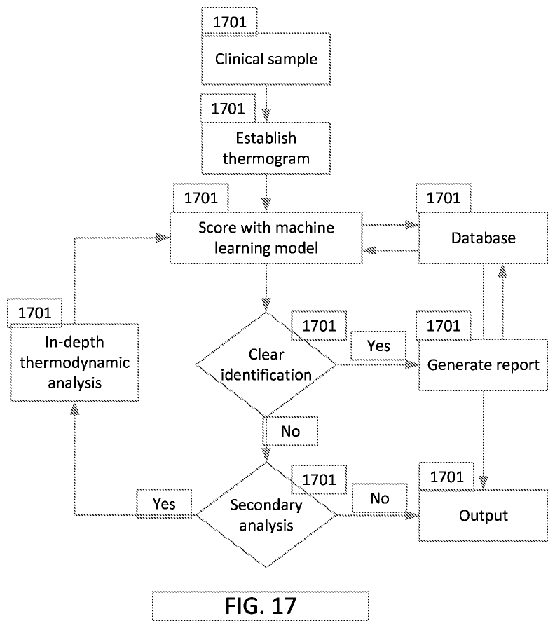

An exemplary process for evaluating, or scoring, clinical samples is illustrated in the flowchart of FIG. 17. A clinical sample is obtained (1701), and a thermogram is established (1702). The thermogram is scored by the machine learning model (1703) and compared with data stored in the database (1704). An assessment of whether there is a clear identification of the ligand(s) is made (1705). If the ligand identification is clear, a report is generated (1706) and the report may be stored in the database (1704) and/or an output is generated (1707). If the ligand identification is not clear, a decision is made whether to perform secondary analysis (1708). If no analysis is performed, an output is generated (1707). In some cases, an in-depth thermodynamic analysis is performed (1709) and the results are scored with the machine learning model (1703). The process then continues as described.

### Example 5

### Drug Development and Clinical Monitoring

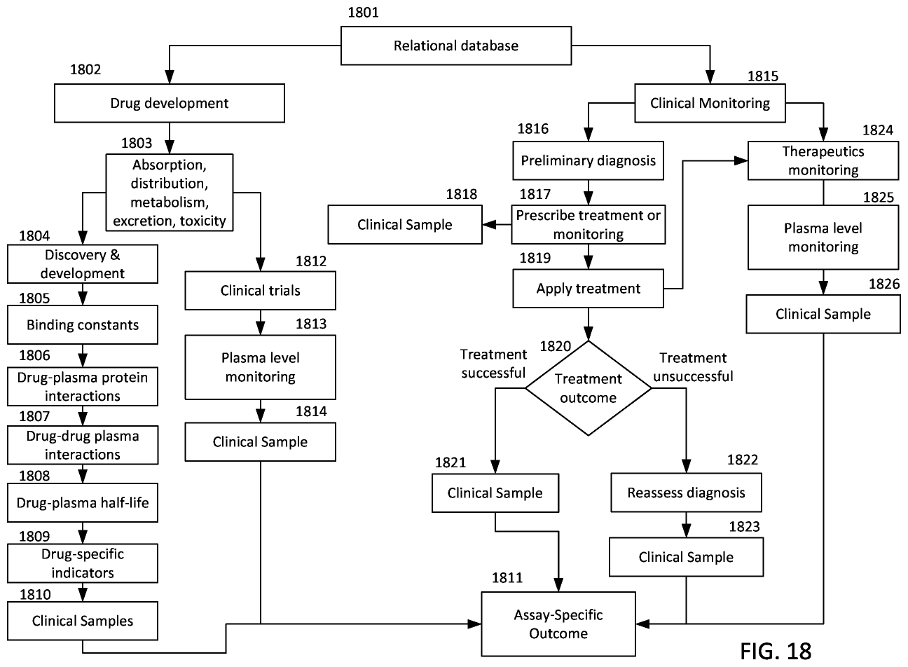

Embodiments of the disclosed relational database have many different uses including, but not limited to drug development and clinical monitoring. Exemplary processes for drug development and clinical monitoring are shown in FIG. 18.

Drug development: Drug candidates are subjected to analysis as discussed in Example 4, and the results are stored in the relational database (1801). Once the drug has been added to the database, the drug development pathway (1802) is followed. Initial stages of drug development including assessing bioavailability, such as absorption, distribution, metabolism, excretion, and toxicity (ADMET) characteristics of the drug candidate (1803). Candidates in the drug discovery and development phase (1804) are preclinical, and samples of the drug candidate would be provided for standard analysis. Standard analysis might include:
binding constants of the drug to plasma proteins (1805);
a detailed breakdown of the drug's interactions with major plasma proteins (1806)
   which proteins the drug interacts with
   effects of the interaction (stabilization, destabilization, etc.)
   stoichiometry of drug binding to the protein;
a detailed breakdown of the drug's interactions with major plasma proteins in the presence of other commonly or co-prescribed drugs (1807);
determination of drug's half-life (1808);
elucidation of drug-specific indicators (1809), such as presence of reactive or unexpected metabolites and interactions.

Clinical samples are assayed (1810) and outcomes determined (1811). Successful drug candidates will move into clinical trials (1812). Plasma level monitoring is determined (1813). Clinical samples may be assayed (1814) and outcomes determined (1811).

The relational database (1801) may be used for clinical monitoring (1815). In some instances, a preliminary diagnosis is made (1816), and a treatment and/or monitoring is prescribed (1817). A clinical sample (1818) may be obtained and analyzed. The treatment is applied (1819), and a treatment outcome is subsequently determined (1820). If treatment appears successful, a clinical sample may be obtained (1821) and analyzed to determine an outcome (1811). If treatment appears unsuccessful, the diagnosis is reassessed (1822). A clinical sample may be obtained (1823) and analyzed to determine an outcome (1811). Clinical monitoring (1815) may include monitoring a therapeutic agent (1824). For example, plasma levels may be monitored (1825). Clinical samples may be obtained (1826) and analyzed to determine an outcome (1811).

### Example 6

### Binding of Chloroquine, DM1, and Tetracaine

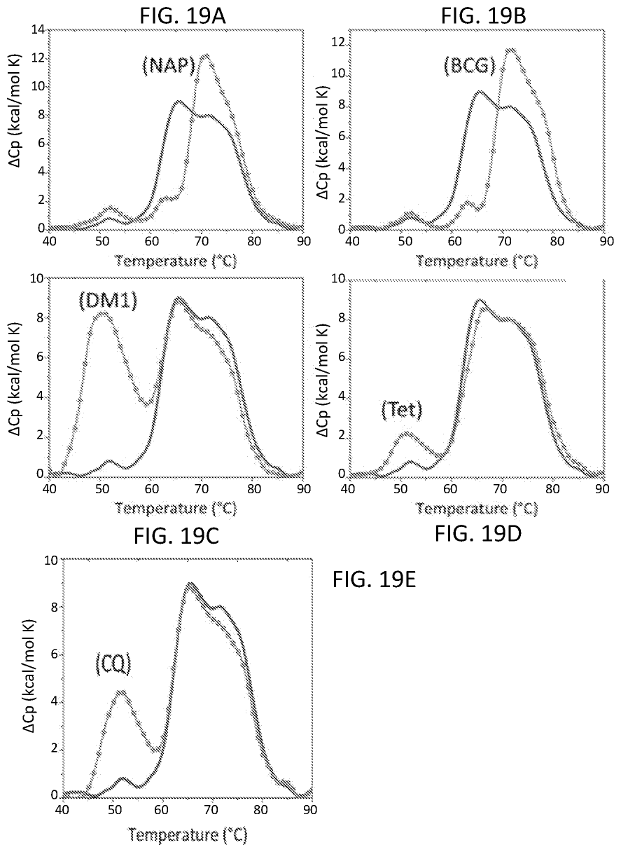

Chloroquine (CQ) is an antimalarial drug. DM1 (also called mertansine) is an antimalarial, bifunctional derivative of chloroquine. Thermograms were measured for plasma in mixtures with 2 mg/mL DM1. The results are shown in FIGS. 19C (DM1) and 19E (CQ); for comparison, FIGS. 19A and 19B show the thermograms for plasma with 2 mg/mL NAP and BCG, respectively. Compared to the control plasma, the measured thermograms were similar at around 62-64° C. where the HSA in plasma transition occurs. However, a significant perturbation was observed at −52° C. for both CQ and DM1. Reproduced in multiple measurements, this peak corresponds to the fibrinogen melting transition in plasma, which is obviously strongly affected by CQ and DM1 binding. There was also a slight decrease on the high temperature side of the main peak, corresponding to IgA, IgG, and IgM, which also suggests minor interactions of those proteins with CQ and DM1.

Previous studies by a collaborator indicated that only 10% of the DM1 circulated in blood while presumably the other 90% remained in the cellular matrix. The results shown in FIG. 16C indicate that the apparently low percentage of circulating drug could be attributable not only to HSA binding but also strong binding to fibrinogen, which effectively keeps the compound in the blood, but not free and therefore unavailable for detection by conventional methods such as equilibrium dialysis.

Tetracaine (Tet), an antiarrhythmia and heart disease drug, was rejected due to the presence of an ester group in the structure and the assumption that the drug would not survive physiological conditions due to the abundant presence of esterases able to break down and deactivate the compound. However, in vivo results showed the compound still retained significant activity after several days, implying that the compound must be protected somehow from esterase activity. A thermogram was obtained for plasma with 2 mg/mL Tet (FIG. 19D). Compared to the control plasma alone, the measured thermogram of the Tet/plasma mixture was only slightly different with a slight increase in Tm from 62-64° C. to 63-65° C. where the HSA in plasma transition occurs. Also, a slight change in the fibrinogen peak was observed at 52° C.

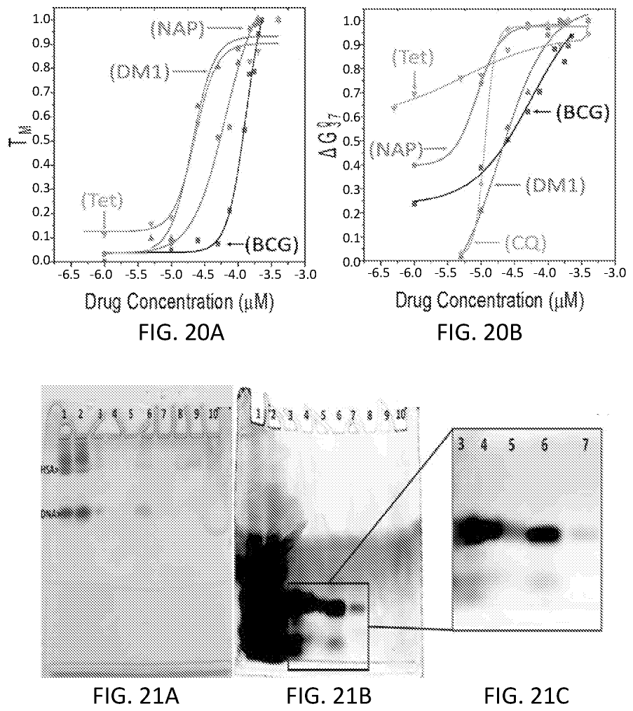

The effects of CQ, DM1 and Tet on HSA were not strongly evident on the thermograms of FIGS. 19C-19E. However, DM1, and Tet binding to HSA is clearly demonstrated in FIGS. 20A and 20B, where the dose response curves of DM1-HSA and Tet-HSA binding are shown. These curves were constructed from titrations of DM1 with HSA. The Tm and ΔG_{cal}(37° C.) were evaluated. The values were normalized against standard HSA and plotted versus ligand concentration to provide the dose curves in FIG. 20A (Tm) and FIG. 20B (ΔG_{cal}(37° C.)). The dose curves of NAP and BCG are shown for comparison. The dose response curve in FIG. 20B indicates that Tet has a high chemical potential of binding, and the binding is energetically favorable and highly specific. The Tm response curve for CQ was omitted from FIG. 20A because CQ did not contribute to a Tm shift of HSA; this is characteristic of a single site binder without stability enhancement. Analysis of the curves provided values for binding constants, stoichiometry and saturation. The results are summarized in Table 2 The Tet results provide a plausible explanation for the unexpected activity of Tet. The drug binds to HSA and fibrinogen in sufficient amounts to protect it from degradation, but allowing access to the compound in blood for target binding.

TABLE 2

| Binding | NAP | BCG | CQ | DM1 | Tet |
|---|---|---|---|---|---|
| Site stoichiometry | 1 | 3 | 1 | 1 | 1 |
| Saturation ratio | 6:1 | 8:1 | 1:1 | 1:1 | 1:1 |
| Constant K_{B} (μM⁻¹), | 1.31 ± | 3.34 ± | 0.409* | 3.81 ± | 4.17 ± |
| average | 1.00 | 1.35 | | 3.31 | 3.32 |
| R² of fit, average | 0.987 | 0.975 | 0.999* | 0.981 | 0.981 |
| Implied number | 6 | 6 | 1 | 1 | 1 |
| of sites | | | | | |

*single trial

### Example 7

### Binding and Capture of DNA

HSA binding of short ssDNA and dsDNA in plasma was investigated. Experiments with ssDNA were performed using ˜1 mg/mL low-salt solution of plasma containing 3 uM 25R ssDNA in 400 μL incubated at 4° C. for at least 24 hours. The incubated sample was added to the capture reagent and the mixture was again incubated at 4° C. overnight. The tube containing the incubated solution sample was then placed under a magnetic field; and the capture reagent along with (presumably) bound ssDNA was pulled to the bottom of the reaction tube. The excess supernatant was removed. To isolate bound components, contents of the tube were subjected to three subsequent washes, each using 100 μL of low-salt buffer. This was followed by two retrieval wash steps, each using 100 μL of high-salt buffer. Supernatant fractions from all washes were collected for subsequent analysis. Results in FIG. 21A clearly show ssDNA was effectively captured using the capture strategy and isolated with a high salt retrieval wash. This is indicated on the gel (lane 6) shown in FIG. 21A. In FIG. 21A, lane 1—plasma+DNA; lane 2—remaining supernatant following capture reagent incubation with plasma; lane 3—first low salt wash; lane 4—second low salt wash; lane 5—third low salt wash; lane 6—first high salt wash; lane 7—second high salt wash (DNA is apparently washed off with high salt); lane 8—HSA standard; lane 9—first supernatant after HSA and capture reagent incubation; lane 10—biotinylated HSA cleaved from the capture reagent.

For binding and capture reactions with dsDNA a ˜1 mg/mL standard plasma low salt solution containing 33.3 uM cy5′-25MER dsDNA in 300 μL was prepared and incubated at 4° C. for at least 24 hours. Conditions differed slightly from those for ssDNA. Since under similar conditions dsDNA appeared to be a relatively weaker binder, and more difficult to visualize, a higher concentration of hot-labeled duplex DNA was employed. Incubated plasma/DNA solutions were subjected to capture procedures carried out in similar fashion as described above for ssDNA. Results are shown in FIG. 21B (lane assignments are the same as FIG. 21A) and 21C where evidently a slight amount of bound dsDNA was effectively captured and washed off the reagent. The results also show a decreased intensity of the DNA band in lanes 3-5, corresponding to the low salt washes with an increased intensity of the DNA in lane 6, corresponding to the first high salt wash. In summary, gel analysis indicated ssDNA and dsDNA binding to HSA was detectable, but weak.

Analysis of thermograms of mixtures of plasma with NAP or BCG presented no problems since the ligands themselves have an essentially insignificant ΔCp over the temperature range of the plasma thermogram (not shown). However, the case is different for mixtures of plasma or HSA with either ssDNA or dsDNA because both ssDNA and dsDNA individually display a very significant ΔCp over the temperature range of the plasma thermogram. In the case of DNA, a direct comparison of the μW versus T curves was preferable. The analysis was required to determine whether thermograms measured for mixtures of plasma or HSA_{B} with DNA were equivalent to the calculated composite curves constructed from the numerical sums of thermograms for the individual components i.e. plasma or HSA and DNA. Essentially, identical measured and calculated composite curves reveal there is little effect of the “interaction” of DNA with plasma or HSA_{B}. At least the interaction is not significant enough to affect the plasma or HSA_{B} thermogram.

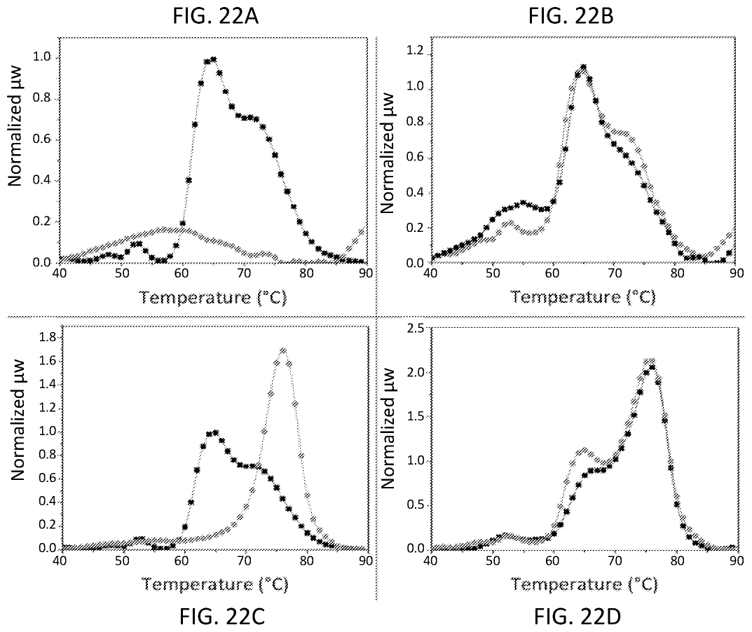

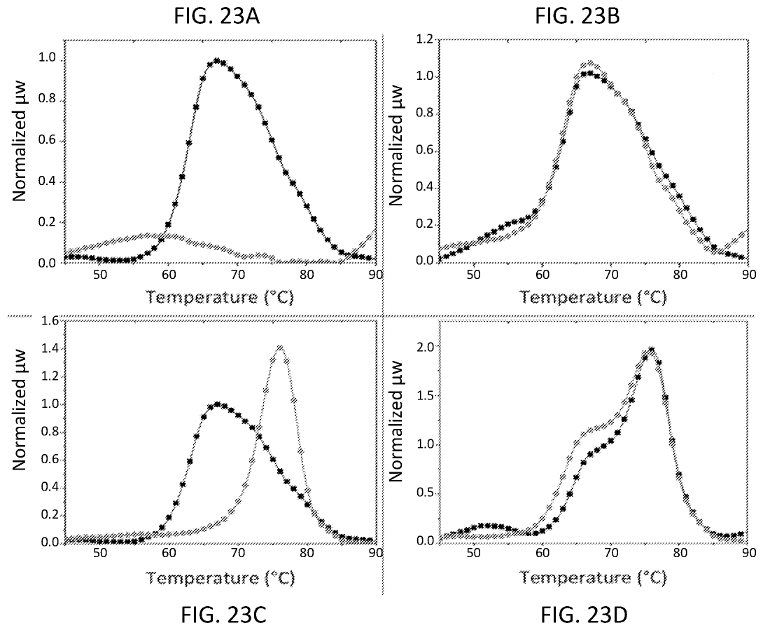

Baseline corrected μW versus T thermograms for the individual components and the measured composite curves of mixtures of plasma and HSA_{B} with ssDNA and dsDNA are shown and compared in FIGS. 22A-22D and 23A-23D. FIGS. 22A-22D are thermograms plotting baseline corrected μW versus temperature for thermograms of plasma alone (▪) and 25 base pair ssDNA alone (●) (22A); measured thermogram of plasma and ssDNA (▪) and thermogram calculated from the sum of the individual thermograms of plasma and ssDNA in FIG. 22A (●) (22B); thermograms of plasma alone (▪) and 25 base pair dsDNA alone (●) (22C); measured thermogram of plasma and dsDNA (▪) and thermogram calculated from the sum of the individual thermograms of plasma and dsDNA in FIG. 22C(●) (22D). FIGS. 23A-23D are thermograms plotting baseline corrected μW versus temperature for thermograms of HSA_{B} alone (▪) and 25 base pair ssDNA alone (●) (23A); measured thermogram of HSA_{B} and ssDNA (▪) and thermogram calculated from the sum of the individual thermograms of HSA_{B} and ssDNA in FIG. 23A (●) (23B); thermograms of HSA_{B} alone (▪) and 25 base pair dsDNA alone (●) (23C); measured thermogram of HSA_{B} and dsDNA (▪) and thermogram calculated from the sum of the individual thermograms of HSA_{B} and dsDNA in FIG. 23C(●) (23D). Calculated composite curves were constructed from individual curves using a linear combination of the respective thermograms of the individual components, measured at exactly the same concentrations as in the mixtures. Experimentally measured composite curves were normalized to the μW versus T thermogram for HSA_{B} alone.

Thermograms (μW versus T plots) for plasma and DNA alone and their mixtures are shown in FIGS. 22A-22D. Those for ssDNA and plasma are shown in FIG. 22A. The thermogram for ssDNA alone displays a small ΔCp that spans the early low temperature region (45-75° C.) of the plasma thermogram. Results of independent experiments with the ssDNA alone and energetic analysis of the sequence (not shown) suggested this transition likely corresponds to melting of a relatively stable intramolecular hairpin loop structure that forms in the short ssDNA oligomer. Displayed in FIG. 22B are the measured thermograms for the ssDNA/plasma mixture and composite thermogram calculated from the sum of the individual thermograms. Two notable observations emerge from the comparison in FIG. 22B. The thermogram for the plasma/ssDNA mixture is not very different from the plasma thermogram alone in FIG. 22A and; the calculated composite thermogram in FIG. 22B is also very close to the measured composite thermogram with only very minor differences. It is tempting to equate these small differences to low level interactions of ssDNA with plasma. If such an interaction does exist, it does not involve substantial changes in thermodynamic stability sufficient to significantly affect the plasma thermogram. Thermograms for ssDNA and HSA_{B} alone are shown in FIG. 23A. Measured and calculated composite curves, just as determined for plasma and ssDNA (FIG. 22A), are shown in FIG. 23B. Again, there are only small differences between measured and calculated composite curves for the mixtures.

Measured and calculated thermograms for mixtures of plasma and ssDNA are nearly quantitatively identical with only minor differences around 48-60° C. and 70-77° C. The major peak on plasma thermograms at −65° C. is attributed primarily to HSA_{B}. The much smaller peak around 53° C. has been attributed to melting of fibrinogen. Since the influence of ssDNA alone has been subtracted out, this higher peak seen at 53° C. could be due to ssDNA interactions with fibrinogen in plasma, but this remains speculative until verification. Regarding the slight difference at −65° C., this region of the plasma thermogram primarily corresponds to melting of immunoglobulins such as IgG and IgA, and may reveal interactions of them with ssDNA

Thermograms of dsDNA and plasma alone are shown in FIG. 22C. Unlike ssDNA, dsDNA displays a significant melting transition that overshadows much of the high temperature region (65-85° C.) of the plasma thermogram. Given that the curves are normalized to the plasma thermogram, apparently under these conditions the DNA has a relatively larger ΔCp compared to plasma. Measured and constructed composite curves are shown in FIG. 22D. Just as seen for ssDNA, calculated composite curves for dsDNA constructed from the sum of the individual thermograms of plasma and dsDNA (measured under precisely the same conditions as in plasma mixtures) were not greatly different. Also consistent with weak, inconsequential (in the thermodynamic sense) binding of dsDNA to HSA, there is a small difference from approximately 60-70° C. which corresponds to the HSA_{B} transition region suggesting perhaps a small contribution from HSA_{B}/dsDNA interactions. Thermograms of dsDNA and HSA_{B} alone are shown in FIG. 23C. The measured and calculated composite curves constructed from the individual thermograms measured under the same conditions are shown in FIG. 23D.

Measured composite curves versus calculated constructed composites in FIGS. 22D and 23D show for both ssDNA and dsDNA, with the minor differences stated above, there is little variation over the entire temperature range. Thus, indicating these ligands interact very weakly in the sense of having an insignificant effect on the thermogram.

NAP and BCG are known to bind HSA and this activity clearly manifests on thermograms of mixtures of the ligands with plasma. The results here, however, showed that although thermograms of plasma alone and mixtures of plasma with DNA were very different, after proper analysis little evidence for binding was actually obtained. Captured DNA (presumably previously associated with HSA in plasma) was detected on gels. From results of independent gel experiments, binding could be detected with an estimated binding constant less than mM (data not shown). Weak binding of DNA to HSA_{B} is consistent with results of AUC experiments that required at least a μM binding constant for detection. In line with this limitation in resolution, our AUC experiments produced no evidence of DNA binding.

Despite low binding activity of ssDNA and dsDNA to plasma, DNA is an ideal example ligand for several reasons. DNA is not really an exogenous ligand per se as similar molecules could actually be encountered endogenously. In this regard DNA is an example of an actual unknown analyte with relatively weak binding to HSA. Experiments with DNA provided a practical test of the efficacy of the capture strategy on such an unknown analyte in plasma. Analysis of DNA plasma thermograms revealed special considerations that must be taken into account for proper analysis of thermograms of the mixtures.

### Example 8

### Effects of HSA Biotinylation and pH

DSC analysis was performed on two commercially available forms of HSA termed HSA₉₉ (99% pure) and HSA₉₆ (96% pure). HSA₉₆ contained a slight amount of contaminant comprised of tight binding globulins and fatty acids (generically termed FA/G-LC). All protein samples were prepared in standard buffer as stock solutions at a concentration of 1.0 mM and stored at 4° C. for at least 24 hours before use. For DSC melting experiments protein samples were 28 μM (˜2 mg/mL), confirmed spectrophotometrically at 280 nm. DSC measurements quantitatively determined how thermodynamic stability of HSA (an indirect measure of HSA structural integrity) was affected by covalent attachment of biotin to different sites; and how thermodynamics of FA/G-LC binding were influenced by biotinylation of lysine residues. DSC analysis was clearly capable of detecting the presence or absence FA/G-LC and differentiate from normal HSA effects of increasing amounts of covalent modification. For both species of HSA, the effect of multiple site modification was a significant temperature shift up with increasing amounts of biotinylation (not shown; Hoang et al., J. Biophys. Chem. 2016, 7(01):9).

Binding of NAP and BCG to differentially biotinylated HSA containing one, five or 10 biotins per molecule was performed as described in the Methods section and examined by DSC. Solutions of HSA or HSA_{B} protein samples for ligand binding experiments each contained NAP or BCG present at different concentrations. Protein concentration was constant in all mixtures at 28 μM (˜2 mg/mL). Protein/Ligand solutions were prepared by adding the desired amount of ligand to the protein solution. NAP or BCG concentrations ranged from one to 225 μM.

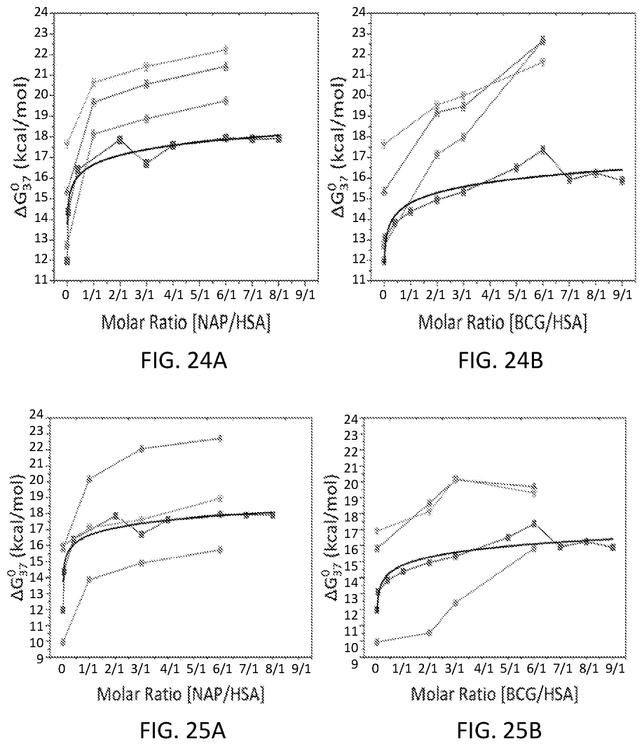

Relative levels of HSA biotinylation were evident on respective thermograms and produced small incremental changes with increased biotinylation. With increased biotinylation, thermodynamic stability incrementally increased up to a ratio of 10:1 biotin:HSA. These results verified sensitivity of DSC to detect the difference in relative amounts of biotinylation and indicated that protein stability is not greatly affected by biotinylation up to a ratio of about 10:1 (biotin:HSA). The results are shown in FIGS. 24A and 24B, respectively. FIG. 24A shows standard HSA bound with NAP (▪), HSA_{B 1:1} with NAP (●), HSA_{B 1:5} with NAP (▴), and HSA_{B 1:10} with NAP (▾). FIG. 24B shows standard HSA bound with BCG HSA_{B 1:1} with BCG (●), HSA_{B 1:5} with BCG (▴), and HSA_{B 1:10} with BCG (▾). The plots in FIGS. 24A-24B demonstrate effects of different levels of HSA modification (via biotinylation) on ligand binding. Semi-quantitative evaluations of thermodynamic quantities, ΔH_{cal} and ΔS_{cal} (ΔH_{cal}/Tm) were made at each ligand concentration. From ΔH_{cal} and ΔS_{cal}, the free-energy at T=37° C., ΔG_{cal} (T)=ΔH_{cal}−TΔS_{cal} was evaluated at each ligand concentration. ΔG_{cal}(37° C.) (=ΔG^{O}₃₇) was plotted versus the molar ratio of NAP (or BCG) in the mixture with HSA as solid lines in FIGS. 24A-24B. Compared to results for normal HSA (also shown in FIGS. 24A-24B) biotinylation resulted in an increase in ΔG_{cal}(37° C.) by as much as ˜6 kcal/mol. Observed differences in the responses for NAP and BCG are likely due to different binding stoichiometry's of NAP (n=1) and BCG (n=3) for their preferable sites on HSA. Curves in FIG. 24A suggest saturation, while those in FIG. 24B do not; entirely consistent with different binding capacities of modified HSA_{B} for the two ligands. Alternatively, it is also possible since BCG has a preference for site I, which has several biotinylatable lysine residues surrounding it, that site I is partially occluded. This forces BCG to bind elsewhere to other less preferential site, i.e. site III. The binding curves in FIGS. 24A-24B whose slope decreases with increasing biotinylation, are consistent with this inference.

Natural HSA samples were prepared at pH 6.0 and pH 8.0 and examined by DSC. These data, shown in FIGS. 25A-25B complement those for normal HSA (pH=7.4). From DSC melting curves thermodynamic parameters for HSA/ligand mixtures were evaluated. FIG. 25A shows standard HSA bound with NAP at pH 7.4 (▪), HSA with NAP at pH 8 (●), HSA with NAP at pH 6 (▴), and HSA with NAP in the presence of 50 μM BCG (▾). FIG. 25B shows standard HSA bound with BCG at pH 7.4 (▪), HSA with BCG at pH 8 (●), HSA with BCG at pH 6 (▴), and HSA with BCG in the presence of 50 μM NAP (▾).

FIG. 25A shows ΔG_{cal}(37° C.) evaluated from DSC melting curves of HSA with mixtures of increasing concentrations of NAP. Curves collected at pH=6.0, pH=7.4, and pH=8.0 are shown and are clearly distinguishable. Since at least a portion of the HSA is presumed to be in alternate isomeric states over this pH range, variations in DSC curves as a function of pH indicate DSC measurements are clearly sensitive to different states of HSA as a function of pH. Also shown in FIG. 25A is the result of titration data collected for mixtures of normal HSA (pH=7.4) with increasing amounts of NAP in the presence of a set amount of BCG (50 μM). This concentration of BCG was chosen to be below the stoichiometry for site I binding. The purpose was to investigate the effect of one ligand on binding of the other for each of the pH dependent HSA structures. Compared to normal HSA (pH=7.4), the titration curve with NAP, in the added presence of BCG, had a slight effect; with ΔG_{cal}(37° C.) approximately equal to that of HSA+NAP mixtures without BCG.

FIG. 25B shows ΔG_{cal}(37° C.) evaluated from DSC melting curves of HSA with various mixtures of increasing concentrations of BCG. Curves collected at pH=6.0, pH=7.4, and pH=8.0 are shown and are clearly distinguishable from normal HSA (pH=7.4). Effect of lowering pH (6.0) was to increase ΔG_{cal}(37° C.) (by as much as ˜5 kcal/mol) over the entire titration range compared to the titration curve for normal HSA (pH=7.4). Meanwhile, the curve at pH=8.0 is lower than that for normal HSA by a slightly smaller amount (˜2 kcal/mol). Effects of titrating normal HSA in mixtures with increasing amounts of BCG in the presence of a set amount of NAP (50 μM) are also shown in FIG. 25B. The plot ΔG_{cal}(37° C.) versus molar ratio HSA/BCG for normal HSA (pH=7.4) was increased in the presence of a single concentration of NAP to the point of being nearly the same curve as observed for HSA with BCG alone, at pH=6.0. The presence of NAP (supposedly bound at site II) apparently affected binding of BCG at site I, at pH=6.0, with the collective effect being a change in global stability of HSA nearly identical to that induced by BCG binding alone to HSA at pH 6.0. Provided this interpretation is reasonable, the results clearly suggest the potential presence of allosteric effects of ligand binding in HSA.

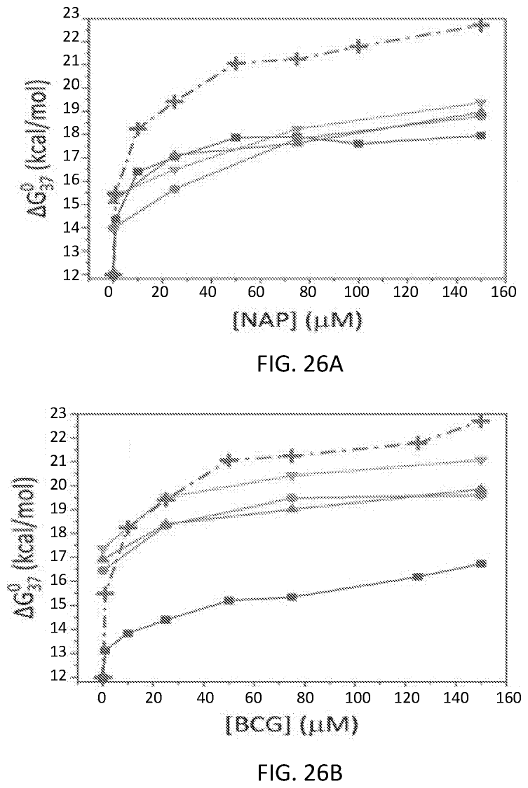

In FIG. 26A, binding curves are displayed for the two-ligand mixtures that contained pre-bound BCG at three different concentrations and in each case with NAP added in a titratable fashion. FIG. 26A shows NAP binding in the presence of BCG: HSA+NAP (▪), HSA+25 μM BCG+NAP (●), HSA+50 μM BCG+NAP (▴), HSA+75 μM BCG+NAP (▾). Also shown in FIG. 26A are the binding curves for NAP alone and the “composite curve” (+) constructed by addition of the individual contributions to binding from NAP and BCG, plus that of HSA alone. Consequently, if pre-binding of BCG, and subsequent binding of NAP were completely independent, the expectation for two-ligand mixtures would be binding curves approximating the composite curve. Examination of the curves in FIG. 26A indicates this is clearly not the case. As seen in FIG. 26A, binding curves of ΔG^{O} versus NAP for NAP alone, and those for NAP in the presence of 25, 50 and 75 μM BCG were not significantly different; and certainly were much less than the composite curve. This indicated NAP binding was greatly diminished by pre-bound BCG and revealed the presence of a strong allosteric effect of BCG on NAP binding.

FIG. 26B shows BCG binding in the presence of varying amounts of NAP: HSA+BCG (▪), HSA+25 μM NAP+BCG (●), HSA+50 μM NAP+BCG (▴), HSA+75 μM NAP+BCG (▾). In FIG. 26B, more pronounced differences were observed between binding curves of BCG and those with HSA pre-bound by NAP. With increased concentrations of pre-bound NAP, binding curves for BCG were much greater than the binding curve of BCG alone. This contrasts the much smaller effect of pre-bound BCG on subsequent NAP binding. Thus, in contrast to the observations in FIG. 26A, pre-bound NAP has a relatively much smaller effect on further BCG binding. As a result, associated allosteric effects of NAP on BCG binding were much less, and binding curves in FIG. 26B approached the composite curve. The results are further summarized in Table 3.

TABLE 3
Binding Chemical Potentials (ΔG^{O}₃₇) (kcal/mol) for two-ligand Binding

| NAP Binding | | | | BCG Binding | | | |
|---|---|---|---|---|---|---|---|
| [NAP] | | | | [BCG] | | | |
| (μM) | HSA_{25BCG} | HSA_{50BCG} | HSA_{75BCG} | (μM) | HSA_{25NAP} | HSA_{50NAP} | HSA_{75NAP} |
| 0 | 14.00 | 15.13 | 15.33 | 0 | 16.45 | 16.92 | 17.38 |
| 25 | 15.65 | 17.12 | 16.50 | 50 | 18.33 | 18.27 | 19.51 |
| 75 | 17.81 | 17.62 | 18.23 | 75 | 19.49 | 18.47 | 20.42 |
| 150 | 18.78 | 18.96 | 19.38 | 150 | 19.61 | 19.29 | 21.08 |
| Error | | | | ±0.2 | | | |

The next series of two-ligand binding experiments compared effects of binding one ligand on subsequent binding of the other for standard HSA and for each of the differentially biotinylated forms of HSA_{B}. For these experiments a pre-bound ligand concentration of 50 μM was chosen because it was the intermediate concentration of those examined in previous two-ligand binding experiments. DSC thermograms measured for the various mixtures revealed sensitivity of the binding-stability linkage and divulged the presence of allosteric interactions in both ligand-bound standard and biotinylated HSA samples. ΔG^{O}₃₇ values determined from DSC thermograms for standard HSA or each of the three differentially modified HSA_{B} molecules in mixtures with a fixed amount (50 μM) of either BCG or NAP, and at titrated concentrations of the other ligand (NAP or BCG) were considered. Results of these experiments for standard HSA and HSA_{B} are summarized in Table 4.

TABLE 4
Binding Chemical Potentials (ΔG^{O}₃₇) (kcal/mol) for two-ligand Binding

| NAP Binding with 50 μM BCG | | | | | BCG Binding with 50 μM NAP | | | | |
|---|---|---|---|---|---|---|---|---|---|
| [NAP] | | | | | [BCG] | | | | |
| (μM) | WT | HSA_{B 1:1} | HSA_{B 1:5} | HSA_{B 1:10} | (μM) | WT | HSA_{B 1:1} | HSA_{B 1:5} | HSA_{B 1:10} |
| 0 | 15.19 | 18.49 | 21.27 | 22.37 | 0 | 16.92 | 20.84 | 23.97 | 23.15 |
| 25 | 17.12 | 21.35 | 23.10 | 23.00 | 50 | 18.38 | 21.76 | 24.12 | 25.04 |
| 75 | 17.62 | 21.92 | 25.45 | 24.03 | 75 | 18.74 | 22.56 | 25.60 | 25.30 |
| 150 | 18.96 | 23.32 | 26.36 | 25.93 | 150 | 19.86 | 22.96 | 25.06 | 24.99 |

| | |
|---|---|
| Error | ±0.2 |

Differences in additive effects of binding BCG and NAP at saturation on global stability of HSA or HSA_{B} were revealed. If pre-bound ligand had no effect, differences would be expected to be nearly equivalent to that of the pre-bound ligand alone. This was clearly not the case for pre-bound BCG with subsequent NAP binding clearly diminished. Thus, a sizable destabilizing allosteric effect is associated with BCG binding (at site I) that affected NAP binding (at site II). Differences in two-ligand binding versus single ligand binding for BCG were seen. Although not totally additive combined effects of pre-bound NAP and subsequent BCG binding were not as strongly linked. Binding by BCG was only slightly affected by pre-bound NAP; and the (allosteric) effect of pre-bound NAP binding on BCG binding was not nearly as large as that for the effect of pre-bound BCG on NAP binding.

In summary, results further exemplified the binding-stability linkage relationship and its persistence (although to a lesser degree) in biotinylated HSA. Results also revealed effects of random site-specific biotinylation of accessible lysine residues on site-specific ligand binding of NAP and BCG. Generally, decreases in ligand binding with increased biotinylation were also observed, but not unexpected. Considering that lysine residues prominently reside in and around critical positions in the binding pockets defined by Sudlow sites I and II, it was not surprising that biotinylation of lysine residues might affect ligand binding (as observed); and that this effect increased with the number of biotins attached (also observed).

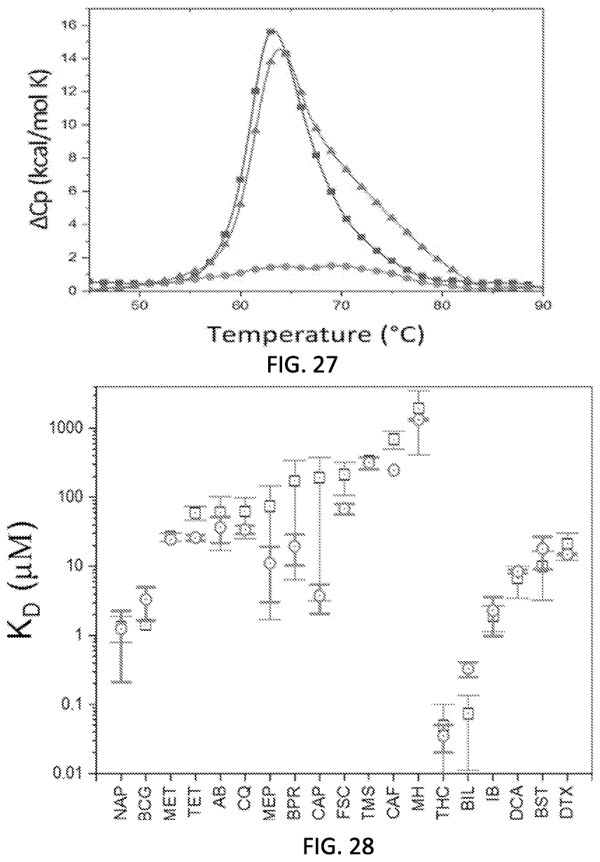

The DSC melting curve of HSA in pH=3.0 buffer (●) is shown in FIG. 27 along with curves for the same sample when returned to pH 7.4 conditions (▴), and a fresh sample of HSA in pH=7.4 buffer (▪). The curve at pH=3.0 has very low intensity suggesting much of the tertiary and secondary structure is largely not intact, or has been degraded, under these conditions. As shown in FIG. 27, this state is apparently reversible, as the normal HSA curve is nearly completely recovered when the pH is returned to pH=7.4. Because of the reversibility, this may be the low pH, so-called E, isomeric form of HSA and not degradation of the sample.

Isothermal titration calorimetry (ITC) is a powerful method used to study the thermodynamics of ligand/protein interactions. A ligand and protein are titrated against one another at a specific temperature, and binding is directly monitored through measurement of the heat exchanged with the environment at each titration point. Under appropriate conditions, ITC measurements and model analysis of the data yields in a single experiment evaluations of the binding reaction enthalpy, ΔH_{B}, binding constant, K_{B}, binding stoichiometry, n, free-energy ΔG_{B}, entropy, ΔS_{B} of binding. The thermodynamic parameters are given by:

$$Δ {H}_{B} ( T ) = Δ H ( {T}_{0} ) + Δ {C}_{P} ( T - {T}_{0} )$$

$$Δ {S}_{B} ( T ) = Δ S ( {T}_{0} ) + Δ {C}_{p} ln ( \frac{T}{{T}_{0}} )$$

$$Δ {G}_{B} ( T ) = Δ H ( {T}_{0} ) - T Δ S ( {T}_{0} ) + Δ {C}_{p} ( T - {T}_{0} - T ln ( \frac{T}{{T}_{0}} ) )$$

T is the temperature and T₀ is a reference temperature. Moreover, ITC experiments performed at different temperatures yield an evaluation of the heat capacity change of the reactions, ΔCₚ. There is a strong correlation between ΔCₚ and buried surface area of the ligand (and protein) upon binding that provides a link between evaluated thermodynamic parameters and structural information. This is because the hydrated water that interacts with the hydrophobic surfaces, and bulk water have very different properties, which leads to a change in heat capacity due to release of water when the ligand binds. Magnitude of ΔCₚ is directly related to the amount of surface area of both the ligand and protein involved in binding. De-solvation of both the ligand and protein upon binding can make either positive or negative contributions to ΔCₚ depending on the types of surface areas involved. If binding involves burial of non-polar surface areas (ΔCₚ<0). For polar surface area (ΔCₚ>0). Heats of binding detected in an ITC experiment are the total heats, which in addition to the heat absorbed or released in the binding event itself, also includes heat effects of dilution of the ligand/protein solution, and mixing of solutions containing different compositions. In order to obtain more accurate heats of binding, these effects can usually be minimized with proper control experiments to identify and eliminate spurious non-specific heat sources. For the purpose of determining ΔCₚ of binding reactions, ITC measurements of selected binding reactions of mixtures of FA and/or test ligands with modified HSA at different temperatures need to be collected.

Samples of natural, highly pure HSA will be biotinylated to provide HSA:biotin ratios of 1:1, 1:5 and 1:10. Biotin levels can be independently verified by streptavidin FITC fluorescent measurements. Modified HSA samples serve as the subjects in studies to be subsequently performed. Samples of isomers of HSA at pH=4, 6 and 8 are prepared. Samples of modified HSA having an average of 25%, 50% or 75% intact disulfide bonds also are prepared. Each of the modified HSAs are prepared for titration experiments with ligands by mixing with eight different concentrations of NAP for a total of 9×8=72 different samples. Identical preparations of HSA_{B} and HSA with eight different concentrations of BCG are prepared for an additional 72 different samples. Additionally, each of the 72 HSA_{B} and HSA samples can be prepared again with each containing a different concentration of NAP with the addition of a constant concentration of BCG, amounting to an additional 72 samples. With analogous samples of HSA_{B} containing eight different concentrations of BCG each with a constant concentration of NAP, another 72 samples can be prepared. In total 288 samples of modified HSA_{B} under different conditions with NAP, BCG or both are prepared. DSC experiments to evaluate the global effects of site-specific ligand binding on overall stability of various modified forms of HSA will be performed.

Additional studies will involve DSC experiments to ascertain how ligand binding to modified HSA (in the nine different forms) is affected by pre-incubation (already bound) with fatty acids (FA) (the more likely state of HSA in vivo). For these studies two medium chain and one long chain FA can be employed. These FA denoted L18, M12, and M8 contain, 18, 12 and 8 carbons, respectively. In general FA, particularly long chain FA, bind HSA with binding constants 10 to 100 times higher than site-specific binding of NAP or BCG. Binding titrations will be performed, and ITC measurements collected on the binding reactions as a function of temperature.

ITC evaluated experimental parameters, ΔH, K_{B} and n will reveal differences in binding parameters for modified HSA compared to normal HSA. Results from ITC experiments conducted with normal HSA will be compared with published reports and assure a common baseline for measuring differences due in binding reactions with modified of HSA.

### Example 9

### Drug Screening

Drug samples were chosen to represent a wide variety of drug classes with clinical utility. Additionally, a few compounds with no known binding activity were also examined, as were several compounds with poor aqueous solubility. In total binding of 28 drug compounds to HSA was examined by obtaining thermograms. The results are summarized in Table 5 where they are compared with literature values for the 28 drug samples. In FIG. 28, results for 19 of the drugs are plotted along with their known literature values with K_{D} values ranging over five orders of magnitude from 10 nm to 1 mM. Excellent agreement is obtained in every case. All evaluated binding constants fall with the error of reported measurements or within a factor less than two of the reported values. Although summarized in Table 5 (not shown in FIG. 28), are those compounds with no reported or observed binding activity.

The analysis also provides additional information regarding effects of functional group modifications of known drugs and associated HSA binding. For example, novel analogs of commercial gadolinium-based contrast agents were analyzed using the DSC method and provided a quantitative measure of specific chemical modifications of existing drugs on HSA binding.

Results obtained for two unique functional, stereochemical derivatives of gadoteric acid (DOTA, sold commercially as Dotarem), denoted as “side” & “corner” are presented in Table 5. Dotarem itself was reported to not have any measurable HSA binding activity. Likewise, the results concurred and no binding was observed for Dotarem. Interestingly however, attachment of a biphenyl thiourea (BP) group to the terminal carboxyl on the standard DOTA structure conferred strong binding affinity (˜10 μM) for HSA. Sensitivity of the analysis procedure able to differentiate relative influence of specific stereochemical orientations of BP attached to DOTA on HSA binding demonstrated the power of the approach.

NBAM-DO3A and BPAM-DO3A are unique functional derivatives of gadoteridol (DO3A, sold commercially as ProHance®). In contrast to the BP-DOTA isomers, DO3A derivatives do not have stereoisomers and therefore are solely functional derivatives, i.e. they only vary through their functional groups. Just as for Dotarem, ProHance® (Bracco) was reported to not display HSA binding activity; and none detected in the analysis. However, addition of a nitrobenzylamine (NBAM) or biphenylamine thiourea (BPAM) conferred appreciable HSA binding and relative differences among them were determined. As shown in Table 2, NBAM displayed a nearly four-fold lower binding constant than BPAM.

The determination that BPAM-DO3A and BP-DOTA display similar binding constants strongly suggests utility of the approach in directing targeted drug design. The ability of quantitative assessment of effects of different functional group modifications on HSA binding can be used to guide design compounds with specifically desired properties. For example, the observed lack of HSA binding activity by ProHance® and Dotarem makes them extremely effective contrast agents ideal for central nervous system imaging. The results argue that expanded scope of use of these and other compounds might be achieved through modifications of critical functional groups and accurate assessment of their HSA binding activities.

Of the 28 compounds examined, 23 were reported to be water soluble. In general, between 40 and 70% of new chemical entities entering the drug development pipeline face bioavailability issues due to poor water solubility. Poor aqueous solubility introduces difficulties for analysis. To address this problem, a novel sample preparation methodology as described in the Methods section was implemented that required minimum amounts of drug sample (milligrams) and avoided entirely the presence of organic solvents in drug/HSA solutions.

A truly remarkable property of HSA, intrinsically related to its transport function, is the ability to accommodate extraordinary levels of ligand binding; actually increasing ligand solubility in plasma up to seven times above the normal solubility limit. This characteristic of HSA forms the basis of the sample preparation methodology which enables preparation of normally insoluble compounds in aqueous solution. With development of this novel process, the universe of potential drug-ligand candidates that can be analyzed is greatly expanded. Effectiveness of the HSA-mediated solubility process was clearly demonstrated for six poorly aqueous soluble compounds denoted in Table 5. Evaluated binding constants were plotted on the right-hand side of FIG. 28 along with reported values of their binding constants for HSA, where excellent agreement was obtained.

TABLE 5
Summary of Drug Binding Parameters

| | | | | | |
|---|---|---|---|---|---|
| | | | | | Literature |
| | Measured | Measured | Average | Average | Standard |
| | K_{D} (10⁻⁶ M) | K_{D} (10⁻⁶ M) | Measured | Literature | Error of |
| Drug | from ΔG⁰₃₇ | From T_{M} | K_{D} (10⁻⁶ M) | K_{D} (10⁻⁶ M) | mean |

Test Compounds

| Bromocresol Green (BCG) | 1.64 | 4.97 | 3.31 | 1.43 | — |
|---|---|---|---|---|---|
| Naproxen (NAP) | 0.21 | 2.27 | 1.24 | 1.33 | 0.54 |
| Chloroquine (CQ) | 29.42 | 38.52 ± | 33.97 | 62.40 | 37.07 |
| | | 4.34 | | | |
| Multihance (MH) | 1290 | 1380 | 1335 | 1948 | 1532 |
| Ablavar (AB) | 21.93 | 51.29 | 36.61 | 60.40 | 43.38 |
| Dotarem (DOTA) | No Binding Detected | | | None Reported | |
| Prohance (PRO) | No Binding Detected | | | None Reported | |
| Magnevist (MAG) | No Binding Detected | | | None Reported | |
| Gadavist (GAD) | No Binding Detected | | | None Reported | |
| Tetracaine (TET) | 28.62 | 23.64 | 26.13 | 59.80 | 13.20 |
| Captopril (CAP) | 2.05 | 5.44 | 3.75 | 191.59 | 188.41 |
| Caffeine (CAF) | 247 | — | 247 | 542.98 | 209.24 |
| Thimerosal (TMS) | 1) 3.56 | 1) 2.70 | 1) 3.13 | 339 | — |
| | 2) 381 | 2) 254 | 2) 317.5 | | |
| Fluorescein (FSC) | 56.16 | 79.82 | 67.99 | 212.03 | 106.16 |
| Metformin (MET) | 2.44 | 1) 1.58 | 1) 2.01 | 26.73 | 3.58 |
| | | 2) 24.52 | 2) 24.52 | | |
| Metoprolol (MEP) | 19.24 | 3.04 | 11.14 | 74.07 | 72.37 |
| Bupropion (BPR) | 1) 0.21 | 1) 0.28 | 1) 0.25 | 172.19 | 165.82 |
| | 2) 10.24 | 2) 28.78 | 2) 19.51 | | |

Novel Compounds

| | | | |
|---|---|---|---|
| DM1157 | 0.90 | 0.92 | 0.91 |
| BP-DOTA (side) | 7.89 | 9.21 | 8.55 |
| BP-DOTA (corner) | 12.37 | 10.45 | 11.41 |
| NBAM-DO3A | 72.04 | 68.38 | 70.20 |
| BPAM-DO3A | 11.62 | 21.54 | 16.58 |

Insoluble or Poorly Aqueous Soluble Compounds

| | | | | | |
|---|---|---|---|---|---|
| Digitoxin (DTX) | 15.15 | 14.56 | 14.86 | 21.27 | 9.12 |
| Ibuprofen (IB) | 1) 3.61 | 0.98 | 2.30 | 1.89 | 0.77 |
| Decanoic Acid (DCA) | 8.96 | 7.88 | 8.42 | 6.70 | 3.30 |
| Δ9- | 0.048 | 0.025 | 0.035 | ≤0.1 | — |
| tetrahydrocannabinol | | | | | |
| (THC) | | | | | |
| B-Estradiol (BST) | 9.02 | 26.86 | 17.94 | 9.98 | 6.74 |
| Bilirubin (BIL) | 0.25 | 0.41 | 0.33 | 0.074 | 0.063 |

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A device for plasma ligand capture, comprising:
a body comprising a substrate material, wherein the body is (i) an elongated body with a polygonal cross-section, or (ii) an annular body;
a poly(methyl methacrylate) (PMMA) coating on at least a portion of a surface of the body; and
a plurality of retrieval moiety molecules covalently bound to the PMMA coating.

2. The device of claim 1, wherein the body is an annular body having an outwardly facing surface and an inwardly facing surface, and the PMMA coating is on at least a portion of the inwardly facing surface.

3. The device of claim 2, wherein:
(i) the annular body has an outer diameter less than an inner diameter of a well of a 96-well plate or less than an inner diameter of a neck of a vial or micro-centrifuge tube; or
(ii) the annular body further comprises an upper annular portion having an outer diameter greater than an inner diameter of a well of a 96-well plate or less than an inner diameter of a neck of a vial or micro-centrifuge tube; or
(iii) the substrate material comprises ferromagnetic steel; or
(iv) any combination of (i), (ii), and (iii).

4. The device of claim 1, further comprising a capture moiety bound to at least one retrieval moiety molecule.

5. The device of claim 4, wherein:
(i) the retrieval moiety molecule comprises streptavidin; or
(ii) the capture moiety comprises biotin covalently attached to a protein capable of binding to a ligand of interest; or
(iii) both (i) and (ii).

6. A method for retrieving a ligand from a plasma sample, comprising:
combining, in a vessel, a capture moiety and a plasma sample comprising or suspected of comprising a ligand, the capture moiety comprising biotin covalently attached to a protein capable of binding to the ligand;
incubating the plasma sample and capture moiety whereby the ligand, if present, binds to the capture moiety to form a conjugate; and
removing the conjugate, if present, from the plasma sample with a device according to claim 1.

7. The method of claim 6, wherein:
(i) the ligand is an exogenous compound or an endogenous component of the plasma sample; or
(ii) the ligand is an exogenous therapeutic compound.

8. The method of claim 6, wherein the protein of the capture moiety is a plasma protein, preferably wherein the plasma protein is human serum albumin (HSA), IgG, fibrinogen, transferrin, haptoglobin, α-1-acid glycoprotein (α-AGP), complement C, or a combination thereof.

9. The method of claim 6, wherein the device comprises the capture moiety, and combining the capture moiety and the plasma sample comprises inserting the device into the plasma sample.

10. The method of claim 6, further comprising:
removing the ligand from the device;
combining the removed ligand with a quantity of plasma or a solution comprising one or more proteins to provide an analysis sample, wherein the plasma or the solution comprising one or more proteins is devoid of the ligand; and
obtaining a thermogram of the analysis sample by differential scanning calorimetry.

11. The method of claim 10, further comprising:
inputting the thermogram into a computer system;
comparing, using the computer system, the thermogram of the analysis sample to (i) a thermogram of a control sample comprising the plasma or the solution comprising one or more proteins, wherein the plasma or the solution is devoid of the ligand, (ii) a reference library of thermograms of samples comprising known ligands and plasma, samples comprising known ligands in solutions comprising one or more proteins, or both (i) and (ii) to provide a comparison; and
determining, using the computer system and based at least in part on the comparison, whether the ligand is present in the analysis sample.

12. The method of claim 11, wherein the ligand is determined to be present in the analysis sample, the method further comprising:
(i) using the computer system and based at least in part on the comparison, determining an identity, a quantity, or an identity and a quantity of the ligand in the analysis sample; or
(ii) analyzing a portion of the ligand removed from the device by chromatography, spectroscopy, gel electrophoresis, or a combination thereof to determine one or more properties of the ligand; or
(iii) both (i) and (ii).

13. The method of claim 12, wherein the plasma sample is obtained from a subject, the method further comprising diagnosing the subject with a disease or condition based at least in part on the identity, the quantity, or the identity and the quantity of the ligand in the plasma sample.

14. The method of claim 12, wherein the plasma sample is obtained from a subject and the ligand comprises an exogenous therapeutic compound, the method further comprising:
comparing, using the computer system, the thermogram of the plasma sample to (i) a thermogram of a control sample comprising plasma or a solution comprising one or more plasma proteins, the control sample being devoid of the exogenous therapeutic compound, (ii) a reference library of thermograms of samples comprising the exogenous therapeutic compound in plasma or the solution comprising one or more plasma proteins, or both (i) and (ii) to provide a comparison;
determining, using the computer system and based at least in part on the comparison, presence of the exogenous therapeutic compound in the plasma sample;
determining, using the computer system and based at least in part on the comparison, a quantity of the exogenous therapeutic compound in the plasma sample; and
determining a bioavailability of the exogenous therapeutic compound or a half-life of the exogenous therapeutic compound in the subject based on a quantity of the exogenous therapeutic compound in the plasma sample and an administered dosage of the exogenous therapeutic compound.

15. A method for drug discovery, comprising:
(a) combining a quantity of a drug candidate with a quantity of a solution comprising one or more plasma proteins to provide an analysis sample;
(b) obtaining a thermogram of the analysis sample by differential scanning calorimetry;
(c) inputting the analysis sample thermogram into a computer system;
(d) comparing, using the computer system, the analysis sample thermogram to a thermogram of a control sample comprising the solution comprising one or more plasma proteins to provide a comparison, the control sample being devoid of the drug candidate;
(e) determining, based at least in part on the comparison, whether the analysis sample thermogram exhibits a perturbation; and
(f) if a perturbation is exhibited,
(i) repeating steps (a)-(e) with one or more additional quantities of the drug candidate; and
(ii) determining, based at least in part on the perturbation, a characteristic of an interaction of the drug candidate with the one or more plasma proteins, wherein the characteristic is a binding constant, reaction enthalpy, binding stoichiometry, binding free energy, binding entropy, or any combination thereof.
